(19) United States
(12) Patent Application Publication
(10) Pub. No.: US 2003/0225011 A1
(43) Pub. Date: Dec. 4, 2003

(54) Phospholipase A2 expression and activity and use thereof for diagnosis, prognostication, prevention and treatment of neural inflammatory and demyelinating disease

(76) Inventors: Samuel David, Dorval (CA); Athena Kalyvas, Pierrefonds (CA)

Correspondence Address: Stephan P. Georgiev, SMART & BIGGAR, Suite 3400, 1000 de la Gauchetiere Street West, Montreal, QC, H3B 4W5, CA

(21) Appl. No.: 10/157,898

(22) Filed: May 31, 2002

(51) Int. Cl. A61K 48/00; C12Q 1/68; C12Q 1/42; A61K 31/66; A61K 31/12

(52) U.S. Cl. 514/44; 514/141; 514/675; 435/6; 435/21

(57) ABSTRACT

The present invention provides methods of preventing and treating neural inflammatory or demyelinating disease, such as multiple sclerosis, via an inhibition of the activity or expression of phospholipase A₂. The invention further relates to methods of identifying phospholipase A₂ inhibitors and their use thereof for the prevention and/or treatment of neural inflammatory or demyelinating disease. An observed increase in the amount of phospholipase A₂ in neural lesions in the EAE animal model system indicates that elevated phospholipase A₂ activity or levels correlate with neural inflammatory or demyelinating disease. Therefore, in a further aspect the invention provides methods for the diagnosis and prognostication of neural inflammatory or demyelinating disease, such as multiple sclerosis.

### FIELD OF THE INVENTION

The invention relates to phospholipase A₂ expression and activity and uses thereof for diagnosis, prognostication, prevention and treatment of neural inflammatory and/or demyelinating disease.

### BACKGROUND OF THE INVENTION

Etiology and Pathogenesis of MS and EAE

Multiple sclerosis is an inflammatory demyelinating disease, which typically strikes young adults, and is characterized by demyelinating episodes ranging from relapsing-remitting to chronic progressive in nature. The lesions are multi-focal and confined to the central nervous system (CNS) which includes the brain, spinal cord and optic nerve. Despite extensive studies, the etiology of the disease still remains obscure and its pathogenesis is not fully understood. The consensus is that unknown environmental agent(s) initiate the disease in genetically susceptible individuals. Several genes are thought to be involved in conferring susceptibility to MS. These include HLA class II (likely the DR2, DQ6 locus) (Tienari, 1994) and the T-cell receptor (TCR) genes (Tienari, 1994). However, a definite set of genetic markers for MS remains unknown. Nevertheless, genetic factors are thought to be important contributors to the onset of the disease because MS shows familial clustering and racial differences in risk (Oger and Lai, 1994; Sadovnick et al., 1996; Ebers, 1996).

A number of environmental factors have also been suspected in MS, such as viral and bacterial infections. Elevated antibody titers against a number of viruses have been reported in the cerebrospinal fluid (CSF) and serum of MS patients (Allen and Brankin, 1993). However, viruses have not been detected in the CNS parenchyma in MS.

MS is studied using the established, generally accepted animal model system of experimental allergic encephalomyelitis (EAE), in for example rodents such as rats and mice (Ruuls et al, 1996; Ewing and Bernard, 1998; van der Meide et al, 1998, Smith et al, 2000. As with MS, EAE is also more easily induced in certain strains of mice and rats.

Target Autoantigens and Cytokines in MS and EAE

An important clue to the pathogenesis of MS is the detection of myelin basic protein (MBP)-reactive T-cells in MS in plaques. Injection of MBP peptides into experimental animals can induce EAE (Richert et al., 1989; Martin et al., 1990). Different regions of MBP are encephalogenic in different animal species, e.g., residues 87-106 in Lewis rats and SJL mice, and 1-9 in PL/J and B10.PL mice. Strong evidence for MBP and additional environmental agents in the pathogenesis of MS comes from studies showing that transgenic mice expressing TCR specific for MBP spontaneously develop EAE but only when exposed to a non-sterile environment (Goverman et al., 1993). Thus, exposure to some infectious agent(s) triggers the breakdown of myelin resulting in the availability of MBP and other myelin components for presentation to the TCR via antigen-presenting cells. T-cells that secrete interferon gamma (IFNγ) with reactivities to MBP, PLP (proteolipid protein), MOG (myelin-oligodendrocyte glycoprotein), and MAG (myelin-associated glycoprotein) have been detected in the CSF of MS patients (Olsson et al., 1990; Sun et al., 1991; Zhang et al., 1993). Anti-PLP antibodies have been detected in about 3% of MS patients (Warren et al., 1994), and PLP has been shown to be encephalitogenic (Tuohy, 1994). Studies also show that MOG may be as effective as MBP or PLP in the pathogenesis of MS and EAE (Adelmann et al., 1995; Johns et al., 1995). Thus, a number of CNS myelin components may serve as target autoantigens.

It is thought that autoantigen specific T-cells sensitized in the periphery migrate into the CNS where they initiate the inflammatory changes leading to CNS tissue damage and functional impairment (Bansil et al., 1995). EAE can be induced by injecting mice with MOG or MBP or by the passive transfer of T-cells from affected animals (Moktarian et al., 1984; Zamvil et al., 1985). The findings to date may be taken to indicate that an initial breakdown of myelin by some yet unknown cause, results in the release of myelin components which are then presented by antigen presenting cells to T-cells with receptor specificity for MBP or other myelin antigens. These interactions result in a variety of immune cell responses leading to antibody production and cytotoxicity.

Proinflammatory cytokines such as IFN-γ, TNF-α and β, IL-12 and IL-1β also increase in the CNS of rats with EAE (Issazadeh et al., 1996) and in the brain in MS (Hofman et al., 1989). IL-2 and IFN-γ mRNA levels were shown to be increased in CSF cells from SJL/J mice during MBP-induced EAE (Renno et al., 1994). IFN-γ increases the severity of the rate of relapse in patients with MS (Panitch et al., 1987). TNF-α and β are present in acute and chronic lesions (Hofman et al., 1989). Furthermore, transgenic mice over expressing TNF-αdevelop a chronic inflammatory demyelination (Probert et al., 1995; Taupin et al., 1997), although other studies on TNF null mice showed similar results (Liu et al., 1998). There are strong similarities in the pathogenesis of MS and EAE (Ewing and Bernard, 1998). As such, EAE is a generally accepted animal model system for MS, and studies on EAE animals have contributed significantly to the understanding of the involvement of the cellular and humoral immune responses in MS (Ewing and Bernard, 1998).

Pathology of MS and EAE Lesions

The CNS lesions in MS and EAE are characterized by widespread focal lesions particularly in perivascular, periventricular and subpial white matter. The pathology varies in acute and chronic lesions. Demyelination is a characteristic feature of acute MS lesions. However, the loss of oligodendrocytes in acute lesions is variable (Bruck et al., 1994; Ozawa et al., 1994). Loss of myelin and oligodendrocytes is more extensive in chronic stages (Prineas et al., 1993; Bruck et al., 1994; Ozawa et al., 1994). The focal lesions also contain inflammatory infiltrates, which consist of T cells and macrophages. In chronic lesions, there is a significant increase in the number of antibody producing plasma cells (Ozawa et al., 1994). CD4⁺ T-cells are found at the edge of the lesions, while macrophages are numerous in and around MS lesions (Traugott et al., 1983; Bo et al., 1994). Activated T-cells are also present in the lesion (Hofman, et al., 1986). Many of these changes in inflammatory cell influx is also seen in EAE lesions in the CNS (Norton et al., 1990; Ewing and Bernard, 1998). However, the inflammatory changes in the CNS rather than demyelination are more prominent in EAE.

Current Approaches in EAE and MS Therapy

Several experimental approaches have been tested in an effort to ameliorate EAE symptoms. Most of these involve immune modulation. These include treatments to block various chemokines or cytokines. Studies performed in blocking chemokines involve the development of anti-MIP-1α, anti-MCP-1 and anti-IP-10 antibodies for treatment of EAE. When the treatment was given before the occurrence of clinical symptoms, anti-MIP-1α antibodies reduced disease incidence by 80% and decreased disease severity from a clinical score of 2.6 in untreated mice to a score of about a 0.5 in treated mice. When this treatment was given after symptoms began, the severity decreased to a score of 1.25. The anti-MCP-1 treatment only had a minimal effect (Karpus et al, 1995). Treatment with anti-IP-10 tested in only a small experimental group decreased incidence by about 65% and reduced the clinical score to a 0.8 compared to a 3.9 in untreated animals (Fife et al, 2001).

Treatments used to block cytokines have also been tested. These involve blocking lymphotoxin, TNF and IFN-γ. In mice given anti-LT/TNFα antibodies before symptoms appeared reduced disease severity from a score of 3.9 in controls to a 0.2 in treated animals (Ruddle et al, 1990). Similar studies using a TNF binding protein completely prevented EAE in animals treated before symptoms were seen. When this treatment was given after symptoms occurred, the treated animals followed a course from a grade 2 to 0, while control animals went from a grade 2 to 3 to 1 (Selmaj, et al, 1998). Treatments performed using anti-IFN-γ antibodies actually worsened disease severity (Leonard et al, 1996).

Other immunomodulatory treatments evaluated were those done to prevent the actions of macrophages and T cells. Animals treated with liposomes (Cl₂MDP) to eliminate macrophages showed a 40% reduction of EAE incidence (Tran, et al, 1998; Bauer et al, 1995, Huitinga et al, 1990). Disease severity was also reduced, from a mean clinical score of 3.4 in controls to a 0.8 in treated animals, when treatment was given before symptoms occurred (Huitinga et al, 1990). Another method to prevent the actions of lymphocytes is to prevent their entry into the CNS by blocking adhesion molecules at the blood-brain barrier. Studies such as these have been performed using antibodies to ICAM-1, LFA-1 and the α4 integrin. Animals treated with anti-ICAM1 or anti-LFA 1 did not show a significant effect in disease reduction. When they were combined, however, their effect reduced a score of 2.5 in control animals to below 0.5 in the treated group (Kawai et al, 1996). Treatments using anti-α4 integrin antibodies reduced clinical incidence by 75% (Yednock et al, 1992) and reduced disease severity from a 1.5 to a 0.5 (Kent et al, 1995). Other experiments in attempts to block proper if T-cell activation and function were also performed. The use of the copper chelator, cuprizone, was used to block IL-2 synthesis and therefore T-cell activation. Treated mice showed decreased disease severity from a score of 4.3 in controls to a 3.3 in mice treated one week before EAE induction. Piperonyl butoxide, an insecticide that is known to deplete T cells delivered before symptoms occurred reduced disease score from a 4.2 in controls to a 2.2. Animals treated after symptoms occurred showed reduced severity to 3.7 (Emerson, et al, 2001).

Oral tolerance has also been evaluated as a treatment for EAE. By feeding animals with myelin antigens, a Th2 response is elicited while Th1 inflammatory responses are reduced. An 80% reduction of EAE incidence was reported in animals fed MBP prior to disease induction. In addition, disease severity was reduced from a maximum score of 4 in control to a 1.4 in treated rats (Popovich et al, 1997). In mice, disease severity was reduced from a 1.6 in control to a 0.6 in treated animals (Meyer et al, 1996). Other methods of switching the Th1 inflammatory cell response to a Th2 cell type response have also been extensively studied. One such treatment is with estrogen. It is known that in pregnant women there is a switch from a Th1 to a Th2 response because of increased levels of this hormone. Mice treated with estrogen showed about a 30% reduction of EAE incidence, a delay of disease onset of about 10 days, and a reduction in disease severity from a 4.5 in untreated animals to a 1.5 (Ito et al, 2001). Mesopram, a type IV phosphodiesterase-specific inhibitor, has also been shown to produce a Th1 to Th2 switch. EAE was prevented in rats treated before the onset of symptoms. Mice treated starting at the first signs of clinical symptoms showed a reduction of a mean clinical score of 4.7 in control animals to a 2.7 in treated animals (Dinter, et al, 2000).

Retinoids, which are ligands of the steroid receptor superfamily, are also thought to favor Th2 cytokine production. They are also thought to increase TGFβ secretion, which is immunosuppressive. When retinamide was given prior to EAE induction, control animals reached a mean clinical score of 3 during relapses, while treated animals went up only to a grade 2 but came down to a 0.75 with no sign of relapse. When retinamide was given after disease symptoms appeared, control animals went from a grade 3.5 to a grade 3 with relapse while treated animals went from a grade 4 to a grade 2 with no relapse (Racke, et al, 1995). Interferon is another molecule thought to serve an immunomodulatory function. Treatment of mice with IFNβ decreases the amount of relapse/mouse from 2.17 in controls to 1.17 in treated animals. Disease severity was also reduced. Control animals progressed from a 3.5 to a 3.8 while treated animals showed a mean clinical score of 3.0 reducing down to a 2.5 (Yu, et al, 1996).

Many signaling pathways are involved in the complex immune reactions seen in EAE and MS. Various kinases are needed to turn-on many of these pathways. Tyrosine kinases mediate the activation of various molecules such as TNFα, prostaglandins (PGE2), and nitric oxide. Tyrosine kinase-blockers have therefore also been evaluated as a possible treatment strategy. These studies have shown about a 60% reduction in incidence of EAE. Also, disease severity was decreased in animals treated before symptoms were seen from a mean clinical score of 3 in controls to a 0.5 in those which received the inhibitor. Mice treated after symptoms occurred reduced severity from a 3 to a 1.5 (Brenner, et al, 1998).

Recent efforts have also focused on decreasing axonal damage in EAE. One way to do this is to reduce the amount of oxidative stress. An inhibitor of inducible nitric oxide synthase (iNOS) given to mice before EAE symptoms appeared decreased symptoms from a 1.3 mean score in controls to a 0.5 in treated animals (Brenner et al, 1997). Metallothinine (MT) is thought to protect cells from reactive oxygen species. Rats treated with MT-II starting at the day of onset of symptoms reduced the score from a 4.5 in controls to a 2 in treated animals (Penkowa and Hidalgo, 2000).

Another way to reduce axonal damage is by blocking glutamate production, which can damage oligodendrocytes and myelin. Experiments using the AMPA/kainate glutamate receptor antagonist NBQX reduced severity from a score of 3 in controls to a 1.5 in treated animals (Smith et al, 2000; Pitt et al, 2000), while MPQX resulted in a greater reduction from a score of 3 to a 0.8. Treating mice during recovery reduced the occurrence of a relapse (Smith et al, 2000).

Of these efforts to develop new treatments for MS, only a few have been approved and are in use. MS therapies currently being used consist of immunomodulatory drugs such as corticosteroids, Interferon beta, and Glatiramer acetate. Corticosteroids have anti-inflammatory and immunosuppressive effects, which also transiently restores the blood-brain barrier (Noseworthy et al, 2000). They shorten the duration of the relapse and accelerate recovery. Since they are only effective as a short-term treatment, they are most commonly used to treat an acute relapse (Anderson and Goodkin, 1998; Bansil et al, 1995). Further,the responsiveness to corticosteroids declines over time, and extended use may lead to adrenal suppression, cardiovascular collapse and arrhythmias. (C. F. Lacy, L. L. Armsrtong, M. P. Goldman, L. L. Lance. Drug information hand book 8^{th} Edition, 2001, 549-551).

Interferonβ has been used as a therapy for patients with active Relapsing/Remitting Multiple Sclerosis (RRMS) since the 1980's. It is recently being used for secondary progressive patients as well. The exact mode of action of this drug is not yet known. It is thought to play an immunomodulatory role by suppressing T cell mediated inflammation (Stinissen et al, 1997). Recombinant IFNβ is available in 3 drugs: IFNβ-1b (Betaseron) and two IFNβ-1a preparations (Avonex and Rebif) (Polman and Uitedehaag, 2000). These drugs reduce rate of clinical relapse. However, neutralizing antibodies develop against these drugs rendering them ineffective with time. Also, flu-like symptoms are a prominent side effect early on in the treatment.

Glatiramer acetate (copaxone) is a synthetic co-polymer of tyrosine, glutamate, alanine and lysine, thought to mimic MBP and thus, block T cell recognition of MBP (Steinman, et al, 1994). This drug is therefore beneficial in RRMS but not progressive MS. This drug also decreases the rate of relapse and appears to be better tolerated by patients than interferon therapy. Further, treatment with this drug may cause cardiovascular problems such as chest pain, flushing and tachycardia, and respiratory problems such as dyspnea. (C. F. Lacy, L. L. Armsrtong, M. P. Goldman, L. L. Lance. Drug information hand book 8^{th} Edition, 2001, 777-779).

Recently, another drug that has been approved for the use in RRMS and secondary progressive MS is mitroxantrone. This drug is used to arrest the cell cycle and prevent cellular division. It is primarily used in leukemias (Rolak, 2001). In MS it reduces relapse rate and increases the length between exacerbations. This drug however has long-term side effects causing cardiac toxicity. Another treatment that has limits to its usefulness is intravenous immunoglobulin. It acts to alter the immune system in a beneficial way and it has shown to cut relapses in half (Rolak et al, 2001). However, the treatments are very expensive.

As discussed above, there are a few moderately effective treatments for RRMS and secondary progressive MS that have shown to reduce both the frequency of the disease and severity of exacerbations. However, problems still exist in treating MS, and there are still no proven treatments, for example, for primary progressive MS. There is therefore a continued need for improved materials and methods for the treatment of neurodegenerative diseases such as MS.

The area of MS diagnosis is significantly less developed, as no measurable biochemical/genetic markers of the disease state exist. As a result, MS diagnosis relies on examining the pathology of the affected tissue by Magnetic Resonance Imaging (MRI) methods. MRI is very costly, and as such its availability is severely limited, typically leading to long waiting lists for testing. Increased cost also limits availability of MRI equipment and expertise to larger communities, thus necessitating travel for those patients residing elsewhere. Further, to justify performing such a costly test, patients are chosen which appear to already exhibit relatively severe symptoms associated with MS, and as such this type of diagnosis is performed significantly later than disease onset, and thus does not provide the opportunity for earlier detection and treatment. There therefore further exists a continued need for improved methods and materials for the diagnosis and prognostication of neurodegenerative diseases such as MS.

### SUMMARY OF THE INVENTION

In a first aspect, the invention provides a method of preventing or treating a neural inflammatory or demyelinating disease in an animal, said method comprising inhibiting the activity of a phospholipase A₂ in the animal.

In another aspect, the invention further provides a method for identifying and/or characterizing a compound for the prevention or treatment of a neural inflammatory or demyelinating disease, said method comprising assaying the activity or expression of a phospholipase A₂ in the presence of a test compound, to identify a compound that inhibits phospholipase A₂ activity or expression, wherein inhibition is indicative that the test compound may be useful for the prevention or treatment of a neural inflammatory or demyelinating disease.

In another aspect, the invention further provides a method of assessing a neural inflammatory or demyelinating disease in an animal, said method comprising:

(a) determining a test level of phospholipase A₂ protein or phospholipase A₂ encoding mRNA or phospholipase A₂ enzyme activity in tissue or body fluid of the animal; and

(b) comparing said test level of phospholipase A₂ protein or phospholipase A₂ encoding mRNA or phospholipase A₂ activity to an established standard; or to a corresponding level of phospholipase A₂ protein or phospholipase A₂ encoding mRNA or phospholipase A₂ enzyme activity in tissue or body fluid of a control animal; or to a corresponding level of phospholipase A₂ protein or phospholipase A₂ encoding mRNA or phospholipase A₂ enzymatic activity in tissue or body fluid obtained from said animal at an earlier time;

wherein an increase in said test level is indicative of the neural inflammatory or demyelinating disease. In an embodiment, the method further comprises the step of assaying the compounds for activity in the prevention or treatment of a neural inflammatory or demyelinating disease. In embodiments, the tissue or body fluid is selected from the group consisting of blood, plasma, cerebrospinal fluid, endothelia, macrophages and lymphocytes.

In an embodiment the above-noted method comprises administering to the animal an effective amount of a phospholipase A₂ inhibitor. In an embodiment the inhibitor is selected from the group consisting of arachidonic acid analogues, benzenesulfonamide derivatives, bromoenol lactone, p-bromophenyl bromide, bromophenacyl bromide, trifluoromethylketones, sialoglycolipids and proteoglycans. In further embodiments, the inhibitor is selected from the group consisting of arachidonyl trifluoromethyl ketone, methyl arachidonyl fluorophosphonate and palmitoyl trifluoromethyl ketone.

In an embodiment the method comprises inhibiting the expression of a phospholipase A₂. In an embodiment the method comprises administering to the animal an effective amount of an inhibitor of phospholipase A₂ expression, such as an antisense molecule. In an embodiment the antisense molecule is a nucleic acid that is substantially complementary to a portion of an mRNA encoding a phospholipase A₂. In an embodiment the antisense molecule is complementary to a portion of a nucleic acid sequence substantially identical to a sequence selected from the group consisting of SEQ ID NO. 1 and SEQ ID NO. 3. In an embodiment the portion of an mRNA comprises at least 5 contiguous bases. In an embodiment the phospholipase A₂ is a mammalian phospholipase A₂, in a further embodiment, human phospholipase A₂. In an embodiment the phospholipase A₂ is a cytosolic phospholipase A₂.

In an embodiment the animal is a mammal, in a further embodiment, a human.

In an embodiment the neural inflammatory or demyelinating disease is Multiple Sclerosis.

In an embodiment the phospholipase A₂ is a cytosolic phospholipase A₂.

The invention further provides uses and commercial packages (comprising the relevant reagent(s) and appropriate instructions to carry out the method) corresponding to the above-mentioned methods.

### BRIEF DESCRIPTION OF THE DRAWINGS

FIG. 1: Schematic illustration of PLA₂ enzyme activity.

FIG. 2: Endothelial cells in EAE lesions express cPLA₂. Spinal cord tissue of mice with EAE at clinical grades 1-4. Arrows indicate cPLA₂⁺ elongated cells in EAE lesions. cPLA₂ positive cells are seen in grades 1-3. No cPLA₂ labeling of endothelial cells is seen in grade 4 micrograph. Slides were counterstained with methyl green which gives a grey staining in the black and white pictures.

FIG. 3: Immune cells in EAE infiltrates also express cPLA₂. cPLA₂⁺ immune cells in the infiltrates were seen at all clinical grades. Arrows point to positive immune cells. Slides counterstained with methyl green.

FIG. 4: Changes in the number of cPLA₂⁺ endothelial cells in EAE lesions. High numbers of endothelial cells, between 45%-85% express cPLA₂ during the earlier stages of the disease, i.e., at grades 1 to 3. The numbers peaked at 85% in grade 3 and reduced to less than 20% at grades 4 and 5.

FIG. 5: Changes in the number of cPLA₂⁺ immune cells in EAE lesions. Between 25% to 50% of the immune cells in the CNS infiltrates were cPLA₂⁺at all clinical grades.

FIG. 6: Histogram showing total numbers of immune cells in EAE lesions. Total number of immune cells infiltrating the CNS at different clinical grades. The number of cells in EAE lesions increases at grades 4 and 5.

FIG. 7: Total number of cPLA₂⁺ immune cells in EAE lesions at different clinical grades. The total number of cPLA₂ positive immune cells increases at later grades of 4 and 5.

FIG. 8: Cell types expressing cPLA₂ in EAE lesions The cell types expressing cPLA₂ in the spinal cords were assessed using double immunofluorescence. GFAP⁺ astrocytes (row 1), CD34⁺ endothelial cells (row 2), Mac-1⁺macrophages (row 3) and CD4⁺ T cells (row 4) were cPLA₂⁺ at and near the EAE lesions. Double labelling of these cells is shown in the column labelled “merge”.

FIG. 9: Incidence of EAE. In the control group, 100% of the mice showed clinical signs of EAE induced paralysis. In contrast to the controls, mice treated with either 2 or 4 mM AACOCF₃ had EAE incidence of 57% and 28%, respectively.

FIG. 10: Clinical course of EAE. Graph showing changes in the clinical course of the disease. EAE was induced in all groups of mice. Mice in the control group (Ctl) that did not receive any treatment reached a peak clinical score of almost 3 at days 12-14 during the first paralytic episode. Compared to the control group, mice treated with 2 and 4 mM AACOCF₃ only reached scores of 1.5 and 0.4, respectively. Furthermore, the control group relapsed into a second paralytic episode between days 26 and 34, while the 4 mM treated group remained unaffected.

FIG. 11: Effect of delayed (i.e. after the peak of the first attack of EAE symptoms) PLA₂ inhibitor treatment of mice. Mice that develop a milder form of the disease, i.e., reach a mean clinical score of 3, while recovering to a grade 2 on day of treatment, show complete recovery and lack of subsequent relapses when treated with 4 mM AACOCF₃ (treat-gr2) In contrast control mice that reach a mean clinical score of 3, while recovering to a grade 2 or less after the first paralytic episode, suffer subsequent paralytic episodes, reaching a mean clinical score of 2.5 (Ctl-gr2).

FIG. 12: Human cPLA₂ DNA sequence (GenBank #: M72393; Sharp et al., 1991).

FIG. 13: Mouse cPLA₂ DNA sequence (GenBank #: M72394; Nalefski et al., 1994)

### DETAILED DESCRIPTION OF THE INVENTION

Although a variety of environmental factors are thought to induce the onset of MS in genetically susceptible individuals, it is proposed herein that these factors likely trigger the activation of a common mechanism that leads to infiltration of immune cells into the CNS, neural tissue damage and myelin breakdown. It is described herein that a likely candidate that could mediate such a common mechanism is the enzyme phospholipase A₂ (PLA₂). One of the metabolic products of PLA₂ is arachidonic acid, which gives rise to eicosanoids such as prostaglandins, thromboxanes and leukotrienes that are potent mediators of inflammatory responses. Another metabolic product of PLA₂ is lysophosphatidylcholine (LPC) which has potent detergent-like properties. Injection of LPC into the CNS and PNS causes myelinolysis (Hall, 1993, Jeffery and Blakemore, 1995; Ousman and David, 2000). LPC is also a chemoattractant for human T cells and monocytes (Ryborg et al., 1994; Prokazova et al., 1998). LPC also induces expression of a number of chemokines and cytokines that are involved in immune cell influx and activation in the CNS (Ousman and David, 2001). Some of these cytokines and chemokines are known to induce the expression of PLA₂. Therefore, LPC produced by PLA₂-mediated hydrolysis of phosphatidylcholine could result in expression of chemokines and cytokines that induce further expression of PLA₂. This cascade could result in inducing severe inflammation (via arachidonic acid)and demyelination (via LPC). Blocking arachidonic acid derivatives such as prostaglandins have been shown to reduce the severity of EAE (Reder et al., 1994). It is proposed herein that blocking a more upstream target i.e., PLA₂ itself would have a profound effect on the induction and progression of EAE as it would block the production not only of arachidonic acid and its derivatives but also the generation of LPC and LPC-induced chemokine and cytokine expression.

Phospholipase A₂

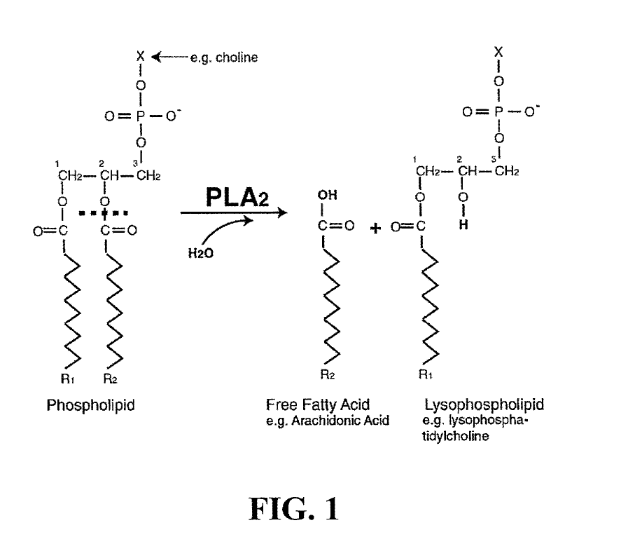

Phospholipase A₂ hydrolyzes the fatty acyl ester bond at the sn-2 position of glycerophospholipids (FIG. 1). The immediate products of a PLA₂-catalyzed reaction are a free fatty acid (e.g., arachidonic acid), and a lysophospholipid (e.g., lysophosphatidylcholine). Phospholipase A₂ has 2 major physiological functions: (1) membrane turnover; (2) potent mediator in the activation of inflammatory processes (Dennis, 1994). Ten different PLA₂ have been identified which fall into two major types: secreted (sPLA₂), and cytosolic (cPLA₂). Various forms of PLA₂ are found in different tissues and cell types or are unique to the venom of reptiles and insects.

Secreted PLA2: Several forms of sPLA₂ exist all of which have molecular weights of about 14 kDa. Group IB sPLA₂ is the pancreatic form that is secreted in digestive juices. It is not expressed in the CNS. Group IIA sPLA₂ is produced by many other cells of the body including neutrophils, thymus, bone a marrow, spleen, astrocytes, Schwann cells, etc., (Kramer et al., 1989; Komada et al., 1989; Ishizake et al., 1989; Wright et al., 1989; Murakami et al., 1990; Nakano and Arita, 1990). Group IIA sPLA₂ is detected in exudates from sites of inflammation or tissue injury such as ascites fluid suggesting that macrophages are a source (Kramer et al., 1989; Trotter and Smith, 1986; Forst et al., 1986; Chang et al., 1987; Seilhamer et al., 1989). Group IIA sPLA₂ from various sources have been purified. It is expressed widely in the brain (Molloy et al., 1998). Another form of sPLA₂, group V is expressed mainly in the heart, lung and placenta, and in very low levels in the brain, except in the hippocampus where it may play a specific physiological role (Molloy et al., 1998). Group X is found mainly in human leukocytes. Groups IA, IIB and III are found only in certain venoms, and group IX in the marine snail (Dennis, 2000). Pro-inflammatory cytokines such as TNF and IL induce expression of sPLA₂ in cultured astrocytes (Oka and Arita, 1991), chondrocytes (Lyons-Giordano et al., 1989) and vascular smooth muscle cells, (Nakano et al., 1990; Arbibe et al., 1997). In addition, human endothelial cells from the umbilical vein express type II sPLA₂ when treated with TNF (Murakami et al., 1993). sPLA₂ require millimolar concentrations of calcium for their activation. U.S. Pat. No. 6,103,469 (Hawkins et al., Aug. 15, 2000) relates to a sPLA₂. The activity of sPLA₂ can be blocked by p-bromophenacyl bromide (Glaser et al., 1993). Other inhibitors are currently being tested by Eli Lilly in preclinical trials in non-CNS models of inflammation (Ogata et al., 2001).

Cytosolic PLA₂: Three forms of cPLA₂ have been identified in recent years. The calcium-dependent form of cPLA₂ (group IV) is found in a variety of mammalian cells and tissues (Glaser et al., 1993). It has a molecular weight of 85 kDa. Group IV cPLA₂ requires micromolar concentrations of calcium and is widely expressed in the brain (Molloy et al., 1998), as well as in neutrophils and endothelial cells (Arbibe et al., 1997; Fujimori et al., 1992; Lautens et al., 1998). It prefers arachidonic acid at the sn-2 position, which means it is capable of selectively releasing arachidonic acid (Glaser et al., 1993). cPLA₂ is phosphorylated and its activation increased by MAP kinase (Lin et al., 1993). Group IV cPLA₂ has been purified from a variety of cellular sources. U.S. Pat. No. 6,242,206 (Choiu et al., Jun. 5, 2001) relates to a cPLA₂.

cPLA₂ expression is increased in neurons in the hippocampus after transient global ischemia (Owada et al., 1994). In addition, mice deficient in cPLA₂ (group IV) are resistant to cerebral ischemia (Bonventre et al., 1997) and MPTP neurotoxicity (Klivenyl et al., 1998). Like sPLA₂, the expression of cPLA₂ in a variety of cells is increased by pro-inflammatory cytokines such as TNF, IFN-γ, IL-1 and CSF-1 (Hulkower et al., 1992; Goppelt-Struebe and Rehfeldt, 1992; Lin, Lin and DeWitt, 1992; Xu et al., 1994; Wu et al., 1994). It can be inhibited by arachidonic acid analogues such as arachadonyl trifluromethylketone (AACOCF₃) and methyl arachidonyl fluorophosphonate (MAFP) (Dennis, 2000; Glaser et al., 1993). Ross et al., (1995) isolated a 180 kDa calcium-dependent form of cPLA₂ from human brain which could be inhibited by bromophenacyl bromide, as well as, AACOCF₃.

Two calcium-independent forms of cPLA₂ have also been isolated from the bovine brain (Hirashima et al., 1992; Farooqui et al., 1997). The 29 kDa form is inhibited by sialoglycolipids, and various proteoglycans (Yang et al., 1994). Another 80-85 kDa calcium independent form of cPLA₂, which exists in multimeric form of 300 kDa has been identified in macrophages. This form can be inhibited by the arachidonic acid analogue, AACOCF₃. Other calcium-independent forms have been identified in myocardial cells and the brush border of the intestine (Murakami, Nakatani Atsumi et al., 1997) but these are not of relevance to the CNS.

The precise physiological role of the various forms of cPLA₂ in the CNS is not known at present. The studies described herein are particularly interested in the ability of cPLA₂ to induce inflammatory responses via production of arachidonic acid. This activity of various forms of cPLA₂ can be effectively inhibited by the arachidonic acid analogues AACOCF₃ and MAFP (Balsinde et al., 1999). Elevated levels of PLA₂ have been detected in MS tissue in an older study (Woelk and Peiler-Ichikawa, 1974), however, this study was done in vitro utilizing post mortem tissue, and thus provides no indication of conditions in living tissue. Another study found no change in the level of secreted PLA₂ activity in MS samples versus controls, and found a decrease in cytosolic PLA₂ activity in samples from MS subjects (Huterer, Tourtellotte and Wherrett, 1995). Furthermore, the downstream products of arachidonic acid and 5-lipoxygenase, such as leukotriene C₄ are elevated in the CSF of MS patients (Dore-Duffy et al., 1991). Levels of prostaglandins, which are derived from arachidonic acid by the action of cyclooxygenase, also correlate with the severity of MS (Dore-Duffy et al., 1986), and blocking these reduces the severity of EAE in mice (Reder et al., 1994). In addition, TNF and IL-β, which are capable of inducing expression of both forms of PLA₂, are elevated in the CSF of patients with MS (Hauser et al., 1990).

LPC Mediates Chemokine and Cytokine Expression and Immune Cell Responses

LPC, another metabolic product of PLA₂, in addition to being a strong myelinolytic agent is also a chemoattractant for T-cells and monocytes and is mitogenic for macrophages (Ryborg et al., 1994; Prokazova et al., 1998). It has been found that injection of LPC into the adult mouse spinal cord leads to the rapid expression of MCP-1, MIP-1α, GM-CSF and TNF-α as determined by RT-PCR (Ousman and David, 2001). The expression of these chemokines and cytokines mediates the rapid influx of T-cells and monocytes into the spinal cord, and to activation of macrophages (Ousman and David, 2000, 2001). These immune cell changes result in rapid demyelination at the site of LPC injection within the spinal cord in 4 days. Previous work of the applicants' laboratory has shown that LPC also induces increased expression of VCAM-1 and ICAM-1 in blood vessels in the mouse spinal cord, as well as, induces a marked opening of the blood-brain barrier (Ousman and David, 2000). These adhesion molecules are important in mediating the extravasation of leukocytes into the CNS parenchyma in EAE and are also expressed in active MS plaques (Lee and Benveniste, 1999; Sobel, Mitchell and Fondren, 1990; Raine and Cannella, 1992).

Described herein are experiments to assess the expression of cPLA₂ in EAE lesions in the spinal cord in C57BL/6 mice. This mouse strain has a naturally occurring null mutation for the major form of sPLA₂ (Group IIA) (Kennedy et al., 1995). Since EAE can be induced in these mice, it is unlikely that sPLA₂ is the only major inducer of the disease. The expression of cPLA₂ was therefore examined in EAE lesions in the spinal cord of C57BL/6 mice using immunohistochemical techniques. As a result, it is shown herein that cPLA₂ is indeed expressed in higher amounts in such lesions. Experiments were then carried out in which the activity of cPLA₂ was blocked using a chemical inhibitor. These experiments revealed that blocking cPLA₂ prevents the onset and progression of EAE.

Demonstrated herein is an increase in cPLA₂ in and around EAE lesions in C57BL/6 mice that have a natural disruption in the sPLA₂ gene. The increase in cPLA₂ was seen in endothelial cells and astrocytes, whose processes surround blood vessels. A high level of expression in endothelial cells was seen just prior to the highest increase in the influx of inflammatory cells into the spinal cord. cPLA₂ expression was also seen in the T cells and macrophages that accumulate at the site of immune lesions in the spinal cord. Previous studies have shown an increase of downstream products of PLA₂ action such as prostaglandins and leukotrienes in the CSF of MS patients (Gallai et al, 1995; Fretland, 1992), however, a role for PLA₂ has not been described prior to the studies described herein. Animal studies using the EAE model to assess the blocking effects of these downstream products have been shown herein to reduce the severity of EAE. A prostaglandin El analogue was shown to delay onset of EAE by a few days and reduce clinical severity from a mean grade of 2.23 in controls to 0.7 in treated rats (Reder et al, 1994). A leukotriene inhibitor, sulfasalazine, also reduced disease incidence in guinea pigs (Prosiegel et al, 1990). A COX-inhibitor, piroxicam, was shown to decrease mean clinical score from a 2.8 in untreated to a 1.5 in treated rats (Weber and Hempel, 1989). In addition, a dual COX/5-lipoxygenase inhibitor was shown to reduce the incidence of EAE (Prosiegel et al, 1989). Provided herein is direct evidence that the use of PLA₂ inhibitors markedly reduces the incidence and severity of EAE. The incidence of EAE using AACOCF3 was reduced by 72% in treated mice. Also, disease severity was reduced from a mean maximal clinical score of almost 3 in control mice to 0.4 in treated mice.

As described herein, the effects of blocking PLA₂ activity are not only immunosuppressive, but also prevent myelin breakdown. The results described herein demonstrate the effectiveness of this inhibitor in an animal model of MS. Therefore, blocking PLA₂ directly can be used as a new therapeutic tool for MS. By blocking PLA₂ upstream of the arachadonic acid metabolites, both the inflammatory cascade and myelin disruption through LPC will be prevented, leading to a potentially effective treatment for MS.

Accordingly, in an aspect, the invention provides a method for the prevention and or treatment of neural inflammatory and/or demyelinating disease in an animal, the method comprising modulating (in an embodiment, inhibiting) the activity and/or expression of a phospholipase A₂ (PLA₂) in the animal. Such a method may comprise administering to the animal an agent capable of modulating PLA₂ activity. In cases involving an inhibition of PLA₂ activity, such an agent is a PLA₂ inhibitor. Such administration may in embodiments occur before, at about the time of, or subsequent to the onset of the disease. An “agent capable of modulating PLA₂ activity” refers to any compound which when introduced into a system comprising a PLA₂ protein, is capable of altering at least one aspect of PLA₂ activity or function. Such an agent may be a ligand of a PLA₂ protein, such as an agonist or antagonist. Such an agent may act directly on a PLA₂ protein or indirectly by modulating a process or activity, which subsequently results in the modulation of PLA₂ activity, or may modulate PLA₂ expression. In certain systems (e.g. in vivo), such an agent may be a prodrug, which is metabolized to an active form at or prior to its arrival at the site of action.

In another aspect, the invention provides a method for the diagnosis and/or prognostication of neural inflammatory and/or demyelinating disease in an animal, the method comprising determining a level of PLA₂ protein or expression or activity of a PLA₂ in a tissue or body fluid obtained from the animal.

In embodiments, the disease is multiple sclerosis and related neural diseases. In further embodiments, the disease is selected from the group consisting of Alzheimer's disease, amyotrophic lateral sclerosis and stroke. In an embodiment, the animal is a mammal, in a further embodiment, a human. In embodiments, the PLA₂ is secreted or cytosolic, calcium dependent or independent. In an embodiment, the PLA₂ is of an average molecular weight of about 14 kDa. In an embodiment, the PLA₂ is of an average molecular weight of about 85 kDa. In an embodiment, the PLA₂ is cytosolic PLA₂ (cPLA₂). In an embodiment, the PLA₂ is a calcium-dependent PLA₂. In embodiments, the PLA₂ is a type IV PLA₂. In embodiments, the method comprises the modulation of both a secreted and a cytosolic PLA₂. In embodiments, the PLA₂ has an activity that generates as a product (a) arachidonic acid (b) lyso-phosphatidylcholine or (c) both (a) and (b).

Chemokines and cytokines, are thought to mediate (play a role in) a variety of disease states. In alternative aspects, the invention relates to methods, uses and commercial packages for immunomodulation (e.g. immunosuppression) and for diagnosis, prognostication, prevention and/or treatment of T-cell mediated diseases, including autoimmune diseases, inflammation, chronic interstitial lung disease, rheumatoid arthritis, inflammatory bowel disease, Crohn's disease, ulcerative colitis, allergy, contact hypersensitivity, psoriasis, systemic lupus erythematosus, osteoarthritis, and diseases mediated by superantigen toxins such as staphylococcal enterotoxin B, and toxic shock syndrome toxin 1.

“Modulation/modulating” as used herein refers to both upregulation (i.e., activation or stimulation (e.g., by agonizing or potentiating)) and downregulation (i.e. inhibition or suppression (e.g., by antagonizing, decreasing or inhibiting)).

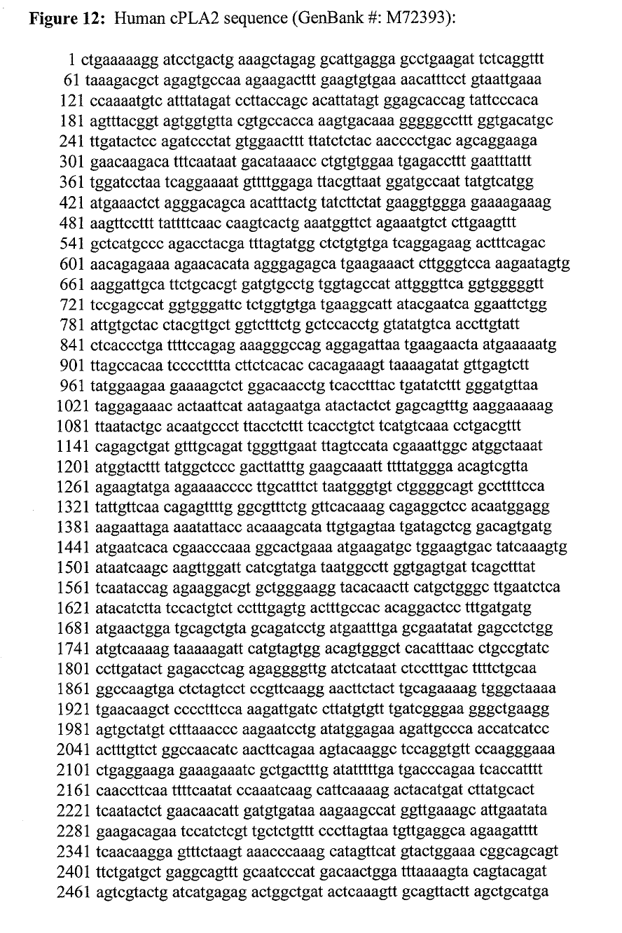

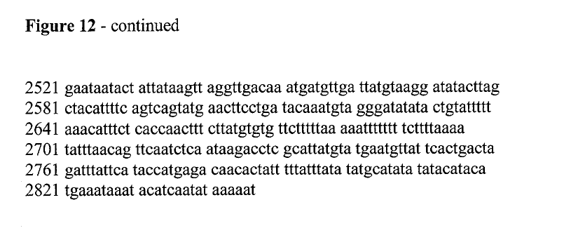

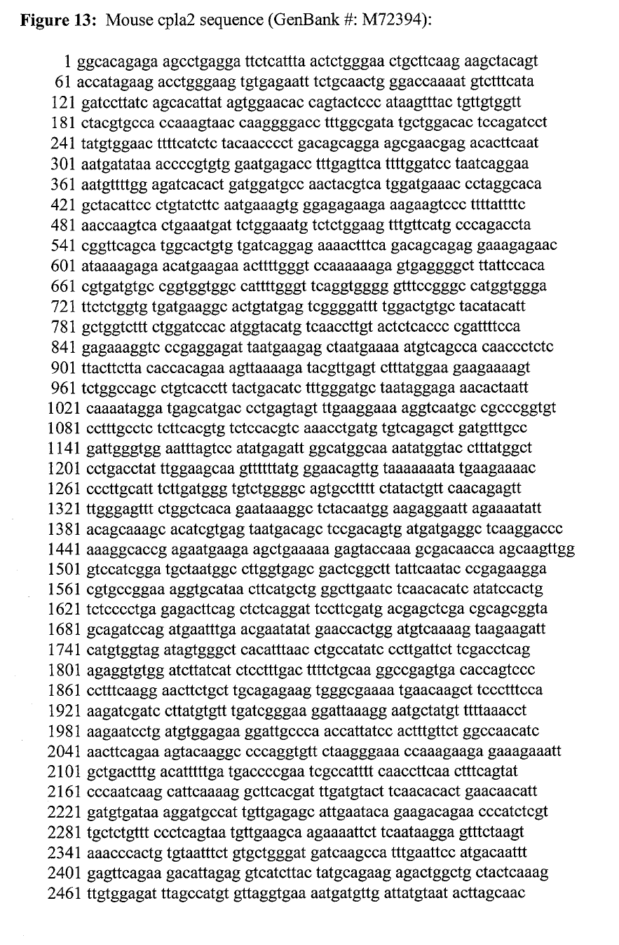

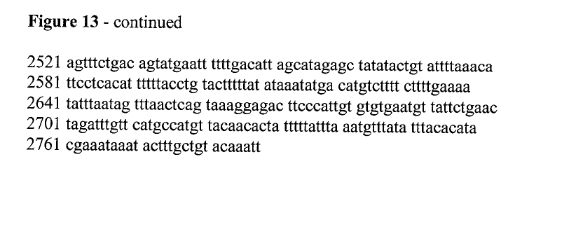

A wide variety of alternative genomic approaches are available to down-regulate the expression of functional PLA₂. For example, in alternative embodiments, transformation of cells with antisense constructs may be used to inhibit expression of PLA₂. Antisense constructs are nucleic acid molecules that may be transcribed to provide an antisense molecule that is substantially complementary to all or a portion of the mRNA encoding PLA₂, so that expression of the antisense construct interferes with the expression of the PLA₂. In an embodiment, the just noted antisense molecule is antisense to a DNA sequence coding PLA₂, in an embodiment, a human PLA₂. Shown in FIG. 12 and SEQ ID NO. 1 is a human DNA sequence encoding a cPLA₂ (Sharp et al., 1991), with the putative coding sequence shown in SEQ ID NO. 1 and the corresponding cPLA₂ protein sequence shown in SEQ ID NO. 2. Shown in FIG. 13 and SEQ ID NO. 3 is a mouse DNA sequence encoding a cPLA₂ (Nalefski et al., 1994), with the putative coding sequence shown in SEQ ID NO. 3 and the corresponding cPLA₂ protein sequence shown in SEQ ID NO. 4. In some embodiments, antisense constructs of the invention may therefore encode five or more contiguous nucleic acid residues substantially complimentary to a contiguous portion a nucleic acid sequence encoding PLA₂, such as an mRNA encoding a PLA₂, or said antisense constructs may encode a sequence of five or more contiguous nucleic acid residues which are antisense to the DNA sequences in SEQ ID NO. 1 and/or SEQ ID NO. 3.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an At indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In alternative embodiments, the invention provides antisense molecules and ribozymes for exogenous administration to bind to, degrade and/or inhibit the translation of PLA₂ mRNA. Examples of therapeutic antisense oligonucleotide applications, incorporated herein by reference, include: U.S. Pat. No. 5,135,917, issued Aug. 4, 1992; U.S. Pat. No. 5,098,890, issued Mar. 24, 1992; U.S. Pat. No. 5,087,617, issued Feb. 11, 1992; U.S. Pat. No. 5,166,195 issued Nov. 24, 1992; U.S. Pat. No. 5,004,810, issued Apr. 2, 1991; U.S. Pat. No. 5,194,428, issued Mar. 16, 1993; U.S. Pat. No. 4,806,463, issued Feb. 21, 1989; U.S. Pat. No. 5,286,717 issued Feb. 15, 1994; U.S. Pat. No. 5,276,019 and U.S. Pat. No. 5,264,423; BioWorld Today, Apr. 29, 1994, p. 3.

Preferably, in antisense molecules, there is a sufficient degree of complementarity to the PLA₂ mRNA to avoid non-specific binding of the antisense molecule to non-target sequences under conditions in which specific binding is desired, such as under physiological conditions in the case of in vivo assays or therapeutic treatment or, in the case of in vitro assays, under conditions in which the assays are conducted. The target mRNA for antisense binding may include not only the information to encode a protein, but also associated ribonucleotides, which for example form the 5′-untranslated region, the 3′-untranslated region, the 5′ cap region and intron/exon junction ribonucleotides. A method of screening for antisense and ribozyme nucleic acids that may be used to provide such molecules as PLA₂ inhibitors of the invention is disclosed in U.S. Pat. No. 5,932,435 (which is incorporated herein by reference).

Antisense molecules (oligonucleotides) of the invention may include those which contain intersugar backbone linkages such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N (CH₃)—CH₂, CH₂—N (CH₃)—N (CH₃)—CH₂ and O—N (CH₃)—CH₂—CH₂ backbones (where phosphodiester is O—P—O—CH₂) Oligonucleotides having morpholino backbone structures may also be used (U.S. Pat. No. 5,034,506). In alternative embodiments, antisense oligonucleotides may have a peptide nucleic acid (PNA, sometimes referred to as “protein nucleic acid”) backbone, in which the phosphodiester backbone of the oligonucleotide may be replaced with a polyamide backbone wherein nucleosidic bases are bound directly or indirectly to aza nitrogen atoms or methylene groups in the polyamide backbone (Nielsen et al., 1991, Science 254:1497 and U.S. Pat. No. 5,539,082). The phosphodiester bonds may be substituted with structures that are chiral and enantiomerically specific. Persons of ordinary skill in the art will be able to select other linkages for use in practice of the invention.

Oligonucleotides may also include species which include at least one modified nucleotide base. Thus, purines and pyrimidines other than those normally found in nature may be used. Similarly, modifications on the pentofuranosyl portion of the nucleotide subunits may also be effected. Examples of such modifications are 2′-O-alkyl- and 2′-halogen-substituted nucleotides. Some specific examples of modifications at the 21 position of sugar moieties which are useful in the present invention are OH, SH, SCH₃, F, OCN, O(CH₂)ₙ NH₂ or O(CH₂)ₙ CH₃ where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic such as cyclobutyl or by another moiety which takes the place of the sugar.

In some embodiments, the antisense oligonucleotides in accordance with this invention may comprise from about 5 to about 100 nucleotide units. As will be appreciated, a nucleotide unit is a base-sugar combination (or a combination of analogous structures) suitably bound to an adjacent nucleotide unit through phosphodiester or other bonds forming a backbone structure.

A number of PLA₂ inhibitors have been described. Such inhibitors include, but are not limited to arachidonic acid analogues such as the arachidonic acid analogues AACOCF₃ and MAFP described above, sialoglycolipids, proteoglycans and p-bromophenyl bromide as noted above, and certain benzenesulfonamide derivatives (Oinuma et al, 1991; European patent application No. 468 054). Further, bromoenol lactone and trifluoromethyl ketones (such as palmitoyl trifluoromethyl ketone, arachidonyl trifluoromethyl ketone) have been reported as inhibitors of Ca⁺⁺-independent PLA₂ (Ackermann et al, 1995) and cPLA₂ (Street et al, 1993) activity as well as bromophenacyl bromide. Accordingly, the invention further provides methods and uses of such compounds for the inhibition of inflammatory and/or demyelinating neural disease, such as MS and related neurodegenerative disease.

In another aspect, the invention relates to the use of a PLA₂ as a target in screening assays that may be used to identify compounds that are useful for the prevention or fr treatment of inflammatory and/or demyelinating neural disease, such as MS and related neurodegenerative disease. In some embodiments, such an assay may comprise the steps of

a) providing a test compound;

b) providing a source of enzymatically active PLA₂; and

c) measuring PLA₂ activity in the presence versus the absence of the test compound, wherein a lower measured activity in the presence of the test compound indicates that the compound is an inhibitor of PLA₂ and is useful for the prevention and/or treatment of inflammatory and/or demyelinating neural disease, such as MS and related neurodegenerative disease.

In another aspect, the invention relates to the use of a PLA₂ as a target in screening assays that may be used to identify compounds that are useful for the prevention or treatment of inflammatory and/or demyelinating neural disease, such as MS and related neurodegenerative disease. In some embodiments, such an assay may comprise the steps of

a) providing a test compound;

b) providing a source of enzymatically active PLA₂;

c) providing a substrate for the PLA₂;

d) assaying the activity of the PLA₂ on the substrate in the presence of the compound, to identify compounds that inhibit the PLA₂, wherein said compound is useful for the prevention or treatment of inflammatory and/or demyelinating neural disease, such as MS and related neurodegenerative disease. In an embodiment the substrate is a phospholipid (e.g. phosphatidylcholine) comprising an arachidonoyl group at the sn-2 position.

The invention also relates to similar assays based on the detection on the expression of PLA₂ and PLA₂ protein levels, which can be detected fro example by immunoassay methods or specific labeling methods, or via a reporter-based assay as noted below.

Such assays may further comprise the step of assaying the compounds for the reduction, abrogation or reversal of EAE symptoms. Such assays may be utilized to identify compounds that modulate expression of the PLA₂ gene, or compounds that modulate the activity of the expressed enzyme.

Screening assays of the invention may also be utilized to identify and/or characterize a compound for inhibiting demyelination. Therefore, the invention further provides a method for identifying and/or characterizing a compound for inhibiting demyelination, said method comprising assaying the activity of a PLA₂ in the presence of a test compound, to identify a compound that inhibits the PLA₂, wherein inhibition is indicative that the test compound may be useful for inhibiting demyelination. In an embodiment, the just noted method may further comprise assaying the compound for inhibition of demyelination.

The above-noted assays may be applied to a single test compound or to a plurality or “library” of such compounds (e.g. a combinatorial library). Any such compounds may be utilized as lead compounds and further modified to improve their therapeutic, prophylactic and/or pharmacological properties for the prevention and treatment of inflammatory and/or demyelinating neural disease, such as MS and related neurodegenerative disease.

Such assay systems may comprise a variety of means to enable and optimize useful assay conditions. Such means may include but are not limited to: suitable buffer solutions, for example, for the control of pH and ionic strength and to provide any necessary components for optimal PLA₂ activity and stability (e.g. protease inhibitors), temperature control means for optimal PLA₂ activity and or stability, and detection means to enable the detection of the PLA₂ reaction product, e.g. arachidonic acid and/or LPC. A variety of such detection means may be used, including but not limited to one or a combination of the following: radiolabelling (e.g. ³²P, ¹⁴C, ³H), antibody-based detection, fluorescence, chemiluminescence, spectroscopic methods (e.g. generation of a product with altered spectroscopic properties), various reporter enzymes or proteins (e.g. horseradish peroxidase, green fluorescent protein), specific binding reagents (e.g. biotin/(strept)avidin), and others.

The assay may be carried out in vitro utilizing a source of PLA₂ which may comprise naturally isolated or recombinantly produced PLA₂, in preparations ranging from crude to pure. Recombinant PLA₂ may be produced in a number of prokaryotic or eukaryotic expression systems, which are well known in the art (see for example U.S. Pat. No. 5,354,677 [Knopf et al., Oct. 11, 1994] for the recombinant expression of cPLA₂. Such assays may be performed in an array format. In certain embodiments, one or a plurality of the assay steps are automated.

A homolog, variant and/or fragment of PLA₂ which retains activity may also be used in the methods of the invention. Homologs include protein sequences, which are substantially identical to the amino acid sequence of a PLA₂, sharing significant structural and functional homology with a PLA₂. Variants include, but are not limited to, proteins or peptides, which differ from a PLA₂ by any modifications, and/or amino acid substitutions, deletions or additions. Modifications can occur anywhere including the polypeptide backbone, (i.e. the amino acid sequence), the amino acid side chains and the amino or carboxy termini. Such substitutions, deletions or additions may involve one or more amino acids. Fragments include a fragment or a portion of a PLA₂ or a fragment or a portion of a homolog or variant of a PLA₂.

The assay may in an embodiment be performed using an appropriate host cell comprising PLA₂ as a source of PLA₂. Such a host cell may be prepared by the introduction of DNA encoding PLA₂ into the host cell and providing conditions for the expression of PLA₂. Such host cells may be prokaryotic or eukaryotic, bacterial, yeast, amphibian or mammalian.

A number of methods for measuring PLA₂ activity may be utilized, such as those described by Reynolds et al. (1994) and Currie et al. (1994) or in U.S. Pat. No. 5,464,754 (Dennis et al., Nov. 7, 1995).

In another embodiment of the invention, a reporter assay-based method of selecting agents which modulate PLA₂ expression is provided. The method includes providing a cell comprising a nucleic acid sequence comprising a PLA₂ transcriptional regulatory sequence operably-linked to a suitable reporter gene. The cell is then exposed to the agent suspected of affecting PLA₂ expression (e.g. a test compound) and the transcription efficiency is measured by the activity of the reporter gene. The activity can then be compared to the activity of the reporter gene in cells unexposed to the agent in question. Suitable reporter genes include but are not limited to beta-D galactosidase, luciferase, chloramphenicol acetyltransferase and fluorescent green protein.

“Transcriptional regulatory sequence” is a generic term that refers to DNA sequences, such as initiation and termination signals, enhancers, and promoters, splicing signals, polyadenylation signals which induce or control transcription of protein coding sequences with which they are operably linked. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since enhancers generally function when separated from the promoters by several kilobases and intronic sequences may be of variable lengths, some polynucleotide elements may be operably-linked but not contiguous. In another embodiment, the construct may comprise an in frame fusion of a suitable reporter gene within the open reading frame of a PLA₂ gene. The reporter gene may be chosen as such to facilitate the detection of its expression, e.g. by the detection of the activity of its gene product. Such a reporter construct may be introduced into a suitable system capable of exhibiting a change in the level of expression of the reporter gene in response to exposure a suitable biological sample. Such an assay would also be adaptable to a possible large scale, high-throughput, automated format, and would allow more convenient detection due to the presence of its reporter component.

The above-described assay methods may further comprise determining whether any compounds so identified can be used for the prevention or treatment of inflammatory and/or demyelinating neural disease, such as MS and related neurodegenerative disease, such as examining their effect(s) on inflammatory cell influx and demyelination in lesions in the EAE animal model system.

In various embodiments, PLA₂ inhibitors, or pharmaceutically-acceptable salts thereof, may be used therapeutically in formulations or medicaments to prevent or treat inflammatory and/or demyelinating neural disease, such as MS and related neurodegenerative disease. The invention provides corresponding methods of medical treatment, in which a therapeutic dose of a PLA₂ inhibitor is administered in a pharmacologically acceptable formulation. Accordingly, the invention also provides therapeutic compositions comprising a PLA₂ inhibitor and a pharmacologically acceptable excipient or carrier. The therapeutic composition may be soluble in an aqueous solution at a physiologically acceptable pH.

The invention provides pharmaceutical compositions (medicaments) containing (comprising) PLA₂ inhibitors. In one embodiment, such compositions include a PLA₂ inhibitor in a therapeutically or prophylactically effective amount sufficient to treat inflammatory and/or demyelinating neural disease, such as MS and related neurodegenerative disease.

The invention further provides a use of a PLA₂ inhibitor or a composition comprising a PLA₂ inhibitor for the prevention and/or treatment of inflammatory and/or demyelinating neural disease, or for the preparation of a medicament for the prevention and/or treatment of inflammatory and/or demyelinating neural disease.

A “therapeutically effective amount” refers to an amount effective, at dosages and for periods of time necessary, to achieve the desired therapeutic result, such as reduction of inflammatory and/or demyelinating neural disease, such as MS and related neurodegenerative disease progression. A therapeutically effective amount of PLA₂ inhibitor may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of inflammatory and/or demyelinating neural disease, such as MS and related neurodegenerative disease onset or progression. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the PLA₂ inhibitors can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. PLA₂ inhibitor) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a PLA₂ inhibitor may be formulated with one or more additional compounds that enhance the solubility of the PLA₂ inhibitor.

In accordance with another aspect of the invention, therapeutic compositions of the present invention, comprising a PLA₂ inhibitor, may be provided in containers or commercial packages which further comprise instructions for use of the PLA₂ inhibitor for the prevention and/or treatment of inflammatory and/or demyelinating neural disease, such as MS and related neurodegenerative disease.

Accordingly, the invention further provides a commercial package comprising a PLA₂ inhibitor or the above-mentioned composition together with instructions for the prevention and/or treatment of inflammatory and/or demyelinating neural disease, such as MS and related neurodegenerative disease.

The positive correlation of PLA₂ expression with EAE indicates that the assessment of the level of PLA₂ protein or a nucleic acid (e.g. an mRNA) encoding PLA₂ or PLA₂ enzyme activity is useful for the diagnosis or prognostication of inflammatory and/or demyelinating neural disease, such as MS and related neurodegenerative disease. PLA₂ mRNA levels may be assessed by methods known in the art such as Northern analysis or RT-PCR (see for example Sambrook et al (1989) Molecular Cloning: A Laboratory Manual (second edition), Cold Spring Harbor Laboratory Press, Cold Spring Harbor, N.Y., USA).

The level of PLA₂ protein or PLA₂ encoding mRNA or PLA₂ enzyme activity may be measured in a variety of tissues and body fluids including but not limited to blood, plasma, cerebrospinal fluid, endothelial cells, macrophages and lymphocytes.

In an embodiment, the level of PLA₂ protein or PLA₂ encoding mRNA or PLA₂ enzyme activity measured in an animal to be tested may be compared to an established standard of PLA₂ protein or PLA₂ encoding mRNA or PLA₂ enzyme activity.

In an embodiment, the level of PLA₂ protein or PLA₂ encoding mRNA or PLA₂ enzyme activity measured in an animal to be tested may be compared to a corresponding level of PLA₂ protein or PLA₂ encoding mRNA or PLA₂ enzyme activity measured in tissue or body fluid of a control animal. In an embodiment, the control animal is an age- and/or weight-matched animal.

In an embodiment, the level of PLA₂ protein or PLA₂ encoding mRNA or PLA₂ enzyme activity measured in an animal to be tested may be compared to a corresponding level of PLA₂ protein or PLA₂ encoding mRNA or PLA₂ enzyme activity measured in tissue or body fluid of the same animal at an earlier time, and such a method is used to prognosticate inflammatory and/or demyelinating neural disease, such as MS and related neurodegenerative disease.

According to a further aspect of the present invention, a commercial package is provided for the diagnosis or prognostication of inflammatory and/or demyelinating neural disease, such as MS and related neurodegenerative disease in an animal. The commercial package comprising means for the assessment of the level of PLA₂ protein or PLA₂ encoding mRNA or PLA₂ enzyme activity in a tissue or body fluid of the animal together with instructions for the diagnosis or prognostication of inflammatory and/or demyelinating neural disease, such as MS and related neurodegenerative disease.

The invention further relates to the use of anti-PLA₂ antibodies for prophylactic, therapeutic, diagnostic and/or prognostic uses. With regard to therapeutic uses, an anti-PLA₂ antibody may be used which is capable of modulating (e.g. inhibiting) the binding and/or catalytic activity of a PLA₂. With regard to diagnostic and prognostic uses, an anti-PLA₂ antibody may be used for detecting PLA₂ and, in embodiments, quantifying the level thereof, in a sample, such as a tissue or body fluid and lymphocytes. Such detection may further be used for imaging methods.

Some anti-PLA₂ antibodies have already been described, such as the anti-cPLA₂ utilized in the Examples below. To prepare such antibodies, a PLA₂ or fragment/homolog/variant thereof may be used to immunize a small mammal, e.g., a mouse or a rabbit, in order to raise antibodies which recognize a PLA₂. An anti-PLA₂ antibody may be either polyclonal or monoclonal. Methods to produce polyclonal or monoclonal antibodies are well known in the art. For a review, see Harlow and Lane (1988) and Yelton et al. (1981), both of which are herein incorporated by reference. For monoclonal antibodies, see Kohler and Milstein (1975), herein incorporated by reference.

Antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monoclonal antibodies may also be in the form of immunoglobulin fragments, e.g., F(ab)′₂, Fab or Fab′ fragments. The antibodies may be of any isotype, e.g., IgG or IgA, and polyclonal antibodies are of a single isotype or a mixture of isotypes.

Anti-PLA₂ antibodies may be produced and identified using standard immunological assays, e.g., Western blot analysis, dot blot assay, or ELISA (see, e.g., Coligan et al. (1994), herein incorporated by reference). The antibodies are used in diagnostic methods to detect the presence of a PLA₂ in a sample, such as a biological sample.

Accordingly, a further aspect of the invention provides a method for assessing an inflammatory and/or demyelinating neural disease, such as MS and related neurodegenerative disease, in an animal, based on detecting the presence of a PLA₂ in a biological sample obtained from the animal, by contacting the biological sample with an antibody capable of recognizing a PLA₂, such that an immune complex is formed, and by detecting such complex to indicate the presence of PLA₂ in the sample.

Those skilled in the art will readily understand that the immune complex is formed between a component of the sample and the antibody, and that any unbound material is removed prior to detecting the complex. It is understood that such an antibody is used for screening a sample, such as plasma, lymphocytes, macrophages, cerebrospinal fluid, urine, saliva, and endo- or epi-thelia for the presence of PLA₂.

For diagnostic applications, the reagent (i.e., an anti-PLA₂ antibody) is either in a free state or immobilized on a solid support, such as a tube, a bead, or any other conventional support used in the field. Immobilization is achieved using direct or indirect means. Direct means include passive adsorption (non-covalent binding) or covalent binding between the support and the reagent. By “indirect means” is meant that an anti-reagent compound that interacts with a reagent is first attached to the solid support. Indirect means may also employ a ligand-receptor system, for example, where a molecule such as a vitamin is grafted onto the reagent and the corresponding receptor immobilized on the solid phase. This is illustrated by the biotin-(strept)avidin system. Alternatively, a peptide tail is added chemically or by genetic engineering to the reagent and the grafted or fused product immobilized by passive adsorption or covalent linkage of the peptide tail.

Such diagnostic agents may be included in a commercial package or kit which also comprises instructions for use. The reagent is labeled with a detection means which allows for the detection of the reagent when it is bound to its target. The detection means may be a fluorescent agent such as fluorescein isocyanate or fluorescein isothiocyanate, or an enzyme such as horseradish peroxidase or luciferase or alkaline phosphatase, or a radioactive element such as ¹²⁵I or ⁵¹Cr.

A further aspect of the present invention is a diagnostic imaging method, which comprises introducing into a biological system, an anti-PLA₂ antibody, which is used in conjunction with an appropriate detection system to identify areas where PLA₂ is present or absent.

The invention further relates to the role of PLA₂ in a variety of in vitro and in vivo inflammatory and/or demyelinating neural disease systems, such as MS and related neurodegenerative disease model systems, such as the EAE model Ad system, and the use of such systems for inflammatory and/or demyelinating neural disease, such as MS and related neurodegenerative disease research. Accordingly, the invention provides a variety of in vitro and in vivo model systems for the study of the mechanisms of the development and progression of inflammatory and/or demyelinating neural disease, such as MS and related neurodegenerative disease, and for the development and characterization of materials and methods for the prevention, treatment, and/or diagnosis of inflammatory and/or demyelinating neural disease, such as MS and related neurodegenerative disease. In an embodiment, such a system comprises a mutation or disruption in a PLA₂ gene or other means of PLA₂ inactivation. In embodiments, the PLA₂ gene encodes a PLA₂ which is cytosolic or secreted, calcium dependent or independent. In an embodiment, the PLA₂ is a cytosolic PLA₂. In an embodiment, both copies of the gene are mutated or disrupted. The system may comprise a transgenic non-human mammal, such as a rodent, such as a mouse.

Applicants have determined that immune cell influx and demyelination at neural lesions correlate with PLA₂ expression and activity. Accordingly, the invention further provides a method of inhibiting immune cell influx and demyelination at neural lesions in an biological system, via inhibiting the activity and/or expression of a PLA₂ in said system. The invention further provides a use of a PLA₂ inhibitor for the inhibition of immune cell influx and/or demyelination at neural lesions in a biological system, or for the preparation of a medicament for the inhibition of immune cell influx and/or demyelination at neural lesions in a biological system. The invention further provides a method of assessing immune cell influx and/or demyelination at neural lesions in a biological system, the method comprising:

(a) determining a test level of PLA₂ protein or PLA₂ encoding mRNA or PLA₂ enzyme activity in said system; and

(b) comparing said test level of PLA₂ protein or PLA₂ encoding mRNA or PLA₂ activity to an established standard;

or to a corresponding level of PLA₂ protein or PLA₂ encoding mRNA or PLA₂ enzyme activity in a control system;

or to a corresponding level of PLA₂ protein or PLA₂ encoding mRNA or PLA₂ enzymatic activity determined in said system at an earlier time;

wherein an increase in said test level is indicative of immune cell influx and/or demyelination at neural lesions.

The invention further provides a commercial package comprising a PLA₂ inhibitor together with instructions for inhibiting immune cell influx and/or demyelination at neural lesions. The invention further provides a commercial package comprising means for the assessment of the level of PLA₂ or PLA₂ encoding mRNA or PLA₂ enzyme activity in a biological system together with instructions for assessing immune cell influx and/or demyelination at neural lesions in biological system.

In embodiments, the above noted biological system is a mammal, in a further embodiment, a human.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

Materials and Methods

Generation of EAE: EAE was induced in female C57BL/6 mice (18-20 g) by subcutaneous injections of 50 kg of myelin oligodendrocyte glycoprotein (MOG_{35-55—}-MEVGWYRSPFSRVVHLYRNGK [SEQ ID NO. 5]) (Sheldon Biotechnology Centre, McGill University) in Complete Freund's Adjuvant (Incomplete Freund's adjuvant containing 1 mg heat inactivated Mycobacterium tuberculosis (Difco Labs)). An intravenous injection of 200 ng of pertussis toxin (List Biologicals) was also administered on days 0 and 2. The mice were monitored clinically for EAE symptoms daily using the following 5-point scale:

Grade 0=normal (no clinical signs).

Grade 1=flaccid tail.

Grade 2=flaccid tail and mild hindlimb weakness (fast righting after mice are placed on their backs).

Grade 3=flaccid tail and severe hindlimb weakness (slow righting after mice are placed on their backs).

Grade 4=flaccid tail and hindlimb paralysis.

Grade 5=flaccid tail, hindlimb paralysis plus forelimb weakness/moribund.

Immunohistochemistry: The mice at different clinical grades were deeply anesthetized and perfused via the heart with 0.1 M phosphate buffer (pH 7.2) followed by perfusion with 4% paraformaldehyde in 0.1 M phosphate buffer (pH 7.2). The spinal cords of the mice were post- fixed for an hour in the same fixative, and then cryoprotected overnight in 30% sucrose in phosphate buffered saline (PBS). Cryostat sections (14 μm) of cross sections of the cervical, thoracic and lumbar spinal cord were incubated in 0.1% H₂O₂ to remove endogenous peroxidases, and then blocked in 0.1% Triton-X 100 and 2% normal goat serum for 5 hours. Tissues were then incubated with an antibody against cPLA₂ (polyclonal rabbit anti-cPLA₂—Santa Cruz Biotech) overnight. Tissue sections were then incubated with a biotinylated goat anti-rabbit antibody and then washed and incubated with the avidin-biotin complex conjugated to horseradish peroxidase (Vectastain kit). The staining was visualized using diaminodibenzidine (Sigma) using protocols described previously (Ousman and David, 2000). Sections were counterstained with 3% methyl green, and then dehydrated in ethanol. The slides were cover slipped in Permount.

Double Immunofluorescence: Cryostat sections of tissue obtained by perfusion as described above were blocked in 0.1% Triton-X 100 and 2% normal goat serum and then incubated overnight with an antibody against cPLA₂ (same as that described above) combined with either antibodies specific for astrocytes (mouse anti-GFAP-Sigma), endothelial cells (rat anti-CD34-BD PharMingen), T cells (rat anti CD4-PharMingen), or macrophages (monoclonal antibody Mac-1). Tissue sections were then washed and incubated with a biotinylated goat anti rabbit secondary antibody combined with the appropriate goat anti-rat/mouse rhodamine-conjugated secondary antibody. Tissue sections were then washed and incubated with fluorescein-conjugated steptavidin. The slides were washed and cover slipped in phenylenediamine containing mounting medium.

Quantification: Counts were done using an ocular grid. For the immunoperoxidase stained sections, two cPLA₂⁺ cell types were counted: round cells (immune cells in the infiltrate at and near EAE lesions) and elongated cells (endothelial cells). Three levels of the spinal cord (cervical, thoracic and lumbar) were quantified for 3 animals in each grade (1-5). Counts were made on three sections at least 45 μm apart. The positive cells were taken as a percentage per lesion.

Treatment of EAE-induced mice with cPLA₂ inhibitors: EAE was induced in C57BL/6 mice as mentioned above. At days 0 and 2 a 50 μl intravenous injection of either 2 mM or 4 mM arachidonyl trifluoromethyl ketone (AACOCF3-Cayman Chemicals) diluted in 1% DMSO buffer was administered. This was followed on alternated days by intraperitoneal injections of 200 μl of the same inhibitor at 2 or 4 mM concentrations until day 24. The mice were scored clinically based on the scoring system described above. Monitoring was done in a blinded fashion so that the person doing the scoring was unaware of the treatment groups.

### Example 2

Expression of cPLA₂ in the Spinal Cord in EAE

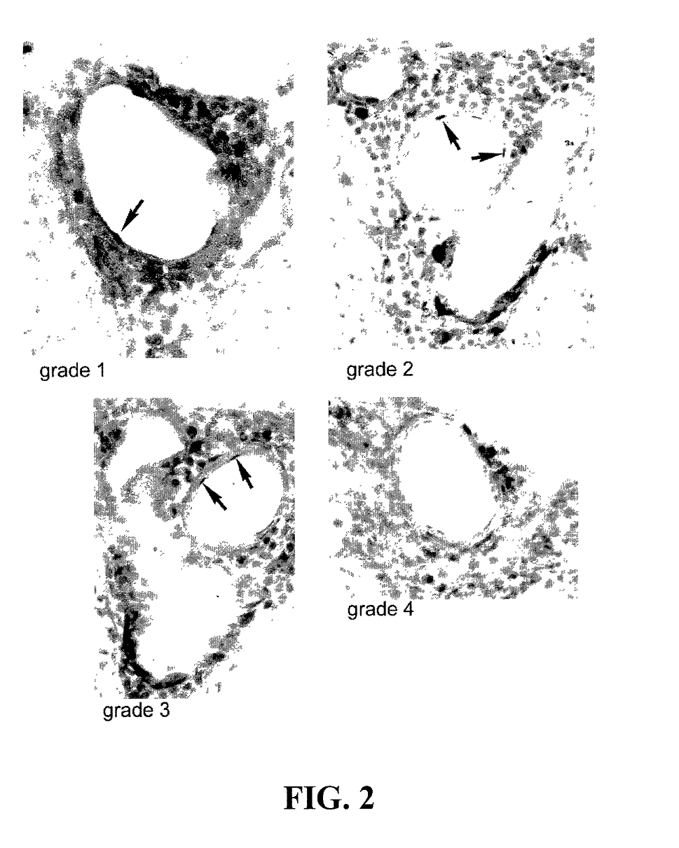

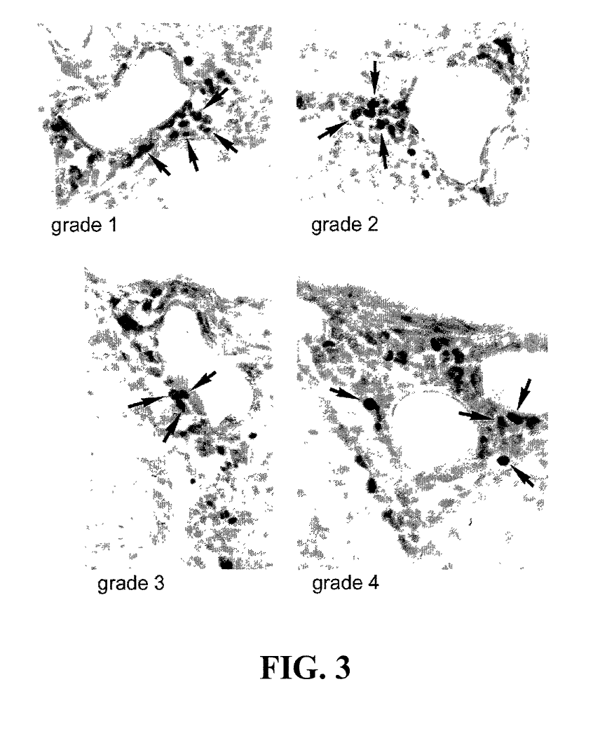

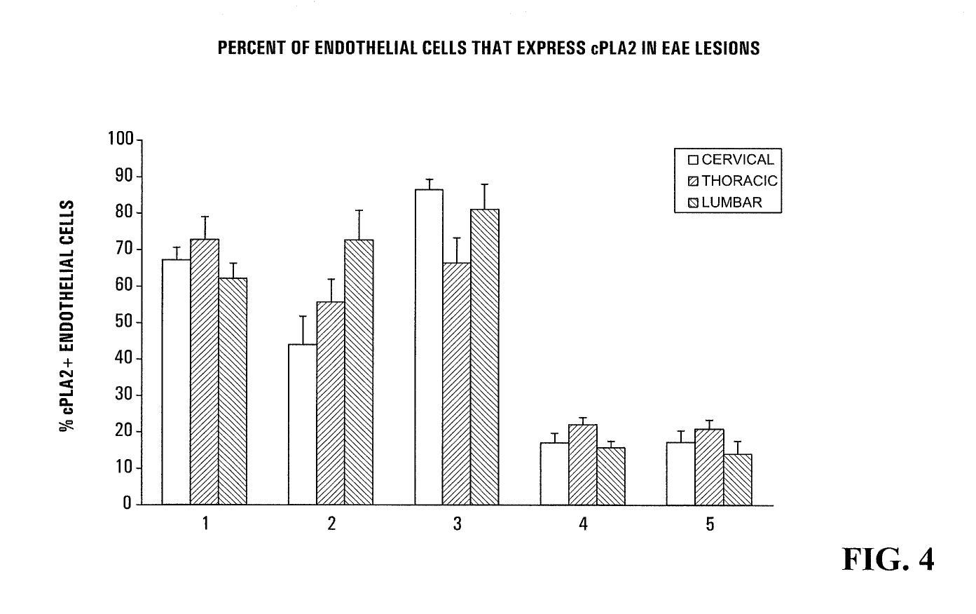

The expression of cPLA₂ in EAE was assessed in the C57BL/6 mouse strain, which has a naturally occurring null mutation for sPLA₂ group IIA (32), the major form of sPLA₂ in the CNS. Therefore, if PLA₂ plays a role in the onset of MOG-induced EAE in C57BL/6 mice, it has to be mediated mainly by cPLA₂. By the immunoperoxidase technique, increased expression of cPLA₂ was observed at the site of EAE lesions in the spinal cord. The labeling occurred in endothelial cells (FIG. 2), as well as immune cells in the CNS inflammatory infiltrates (FIG. 3). The percentage of cPLA₂⁺ endothelial cells ranged from 70% to 85% between clinical grades 1-3, and decreased to about 20% at clinical grades 4 and 5 (FIG. 4).

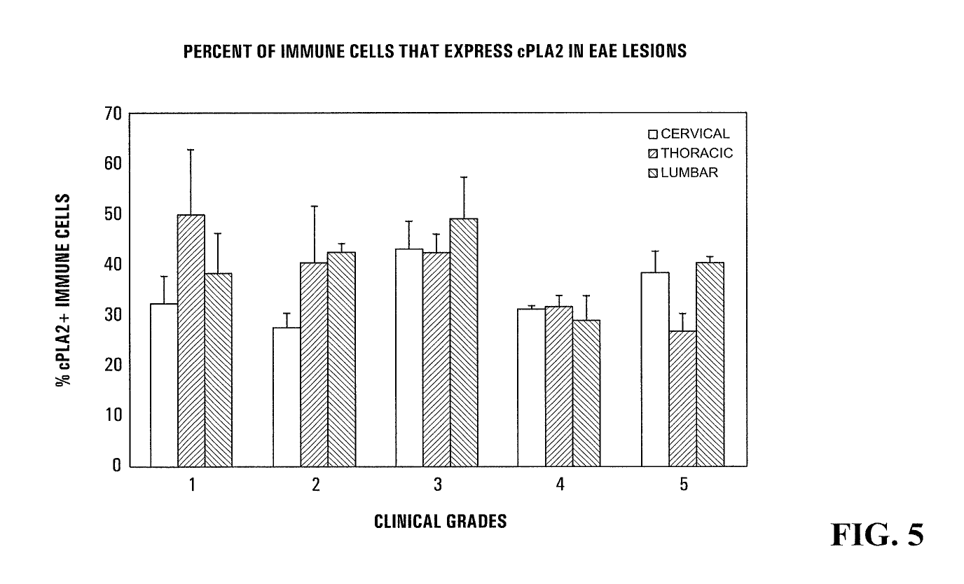

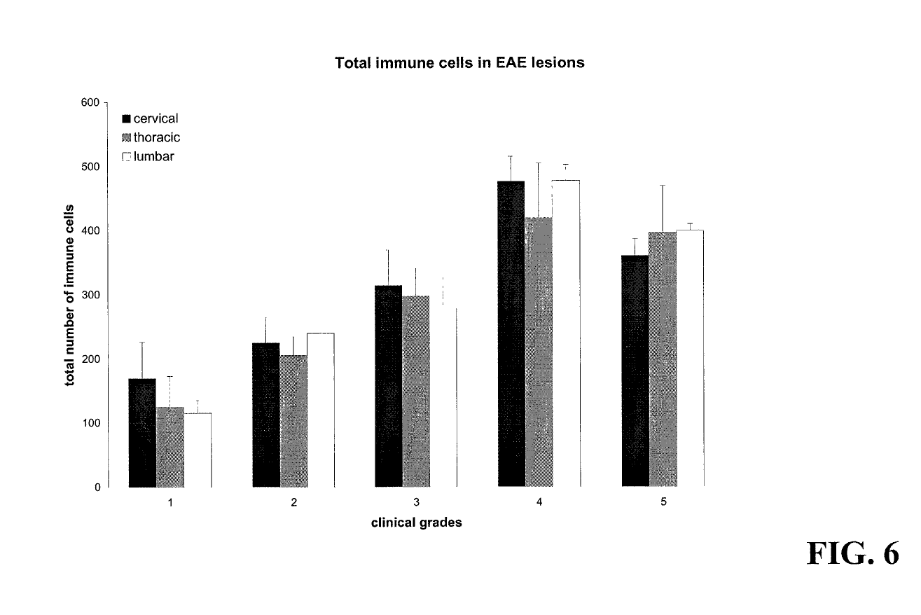

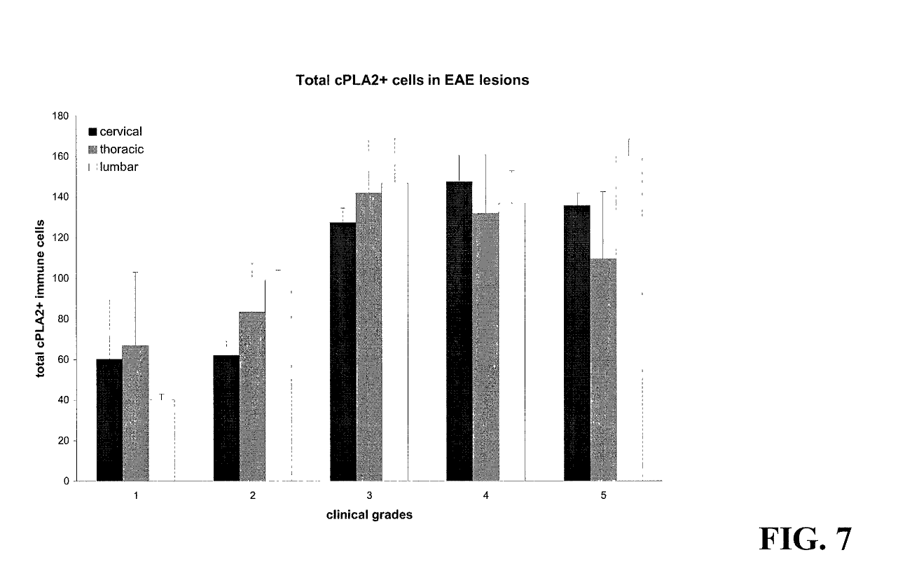

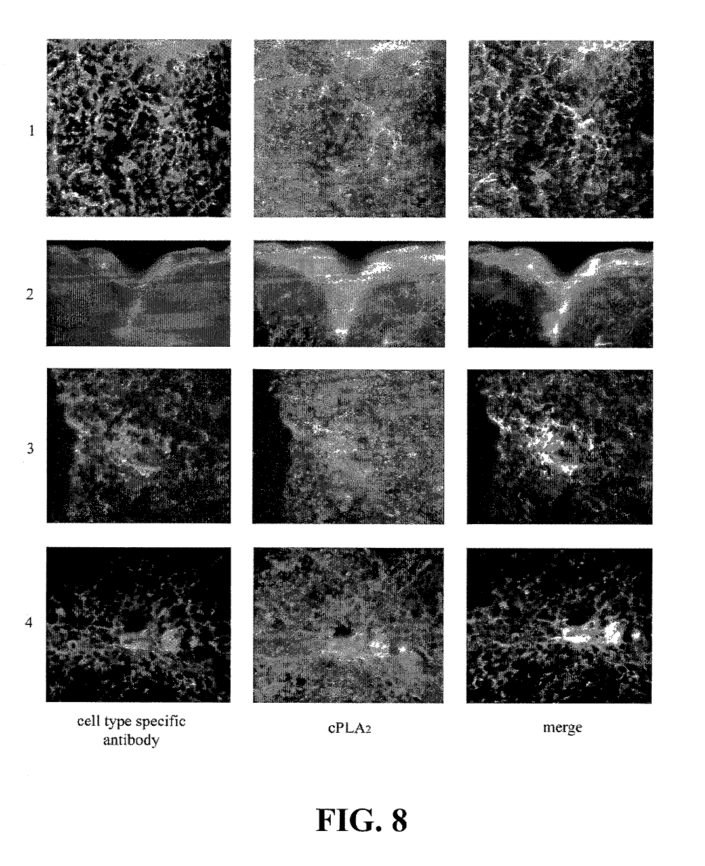

The percentage of cPLA₂⁺ round cells in the immune cell infiltrates in EAE lesions remained at around 30%-50% in all clinical grades (FIG. 5). However, since the total number of cells in the infiltrates increases with increasing clinical grades and with higher inflammatory scores (FIG. 6), the total number of cPLA₂ cells in the spinal cord increased with increasing severity of the disease (FIG. 7). Double-immunofluorescence labeling studies indicate that T cells, macrophages and astrocytes near EAE lesions express cPLA₂ (FIG. 8). These results show that the highest number of cPLA₂⁺ endothelial cells are seen at grades 1-3 which precedes the period of highest influx of inflammatory cells at grade 4.

### Example 3

Blocking with a cPLA₂ Inhibitor Prevents the Onset of EAE

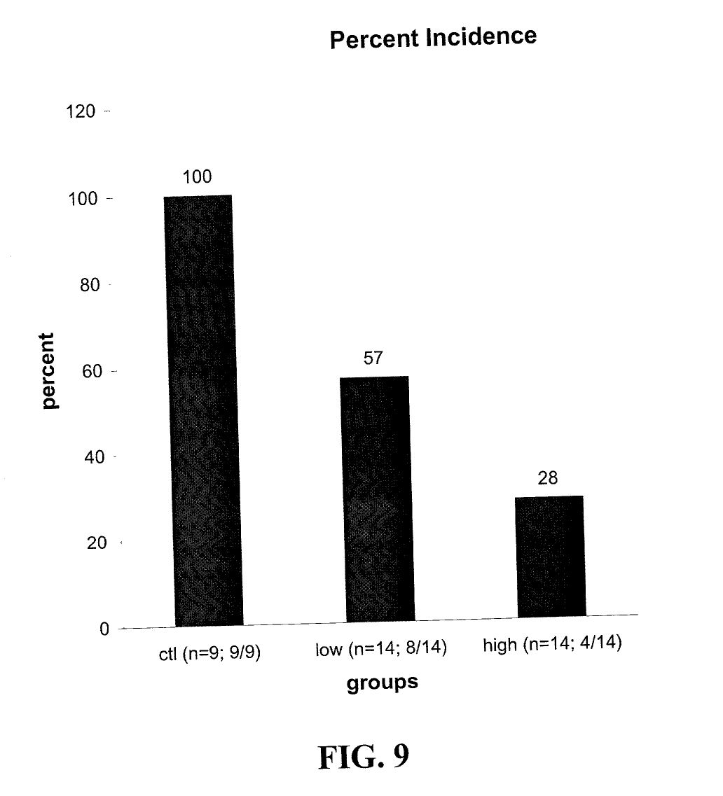

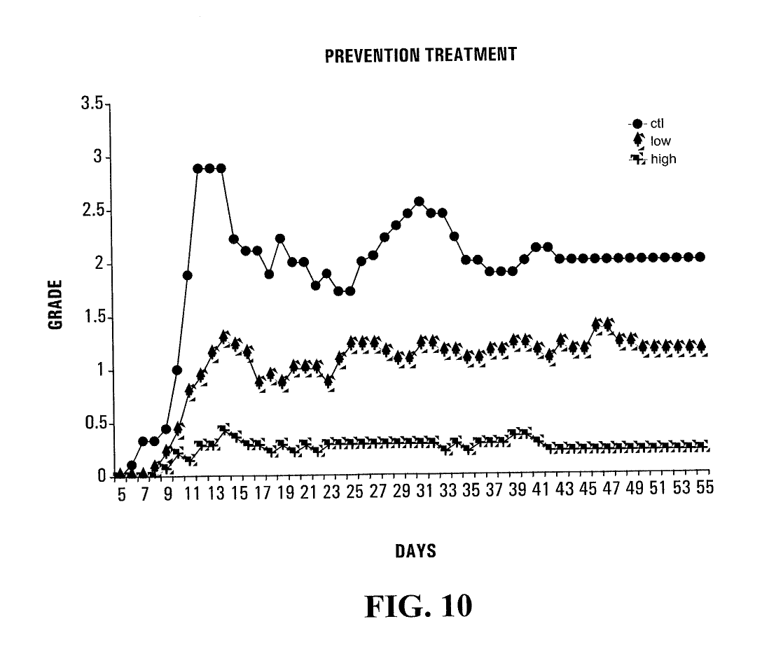

To assess if cPLA₂ is important for the onset of EAE we blocked it using a chemical inhibitor. C57BL/6 mice were treated with the cPLA₂ inhibitor AACOCF₃ on the day of immunization and on day 2 with 50 μl of 2 or 4 mM AACOCF₃ intravenously, followed by intraperitoneal injections of the inhibitor (200 μl at 2 or 4 mM) on alternate days until day 24. Mice were monitored clinically using the scoring scale described above. Treatment with the inhibitor resulted in a remarkable reduction in the onset and progression of EAE. 100% of the vehicle-treated control mice got EAE, while 57% of the 2 mM treated and only 28% of the 4 mM treated groups got EAE (FIG. 9). The progression of the disease was also markedly reduced as shown in FIG. 10. Vehicle-treated controls reached an average maximum clinical score of 2.9 at 12-14 days, while the 2 mM and 4 mM treated groups reached scores of 1.5 and 0.4, respectively (FIG. 10). Unlike the controls, which relapsed into a second paralytic episode between days 25-34, mice treated with 4 mM AACOCF₃ remained unaffected (FIG. 10). The analysis was carried out blind, so that the person doing the clinical scoring was unaware of the treatment groups. The treatment is well tolerated in that the animals do not show any side-effects. The body weight and food-intake of treated mice were unaffected compared to controls at 35 days after induction of EAE. These results provide very strong evidence that blocking PLA₂ has a profound effect in the prevention of EAE.

### Example 4

Delayed Treatment of EAE-Induced Mice with a cPLA₂ Inhibitor

Materials and Methods:

EAE was induced in C57BL/6 mice as described above. A 50 μl intravenous injection of either 4 mM AACOCF₃ diluted in 1% DMSO containing buffer or vehicle (1% DMSO containing buffer) was administered on days 14, 16, 18 and 20 after induction of EAE, when animals began to remit. The mice were scored clinically in a blinded fashion as mentioned above.

Results:

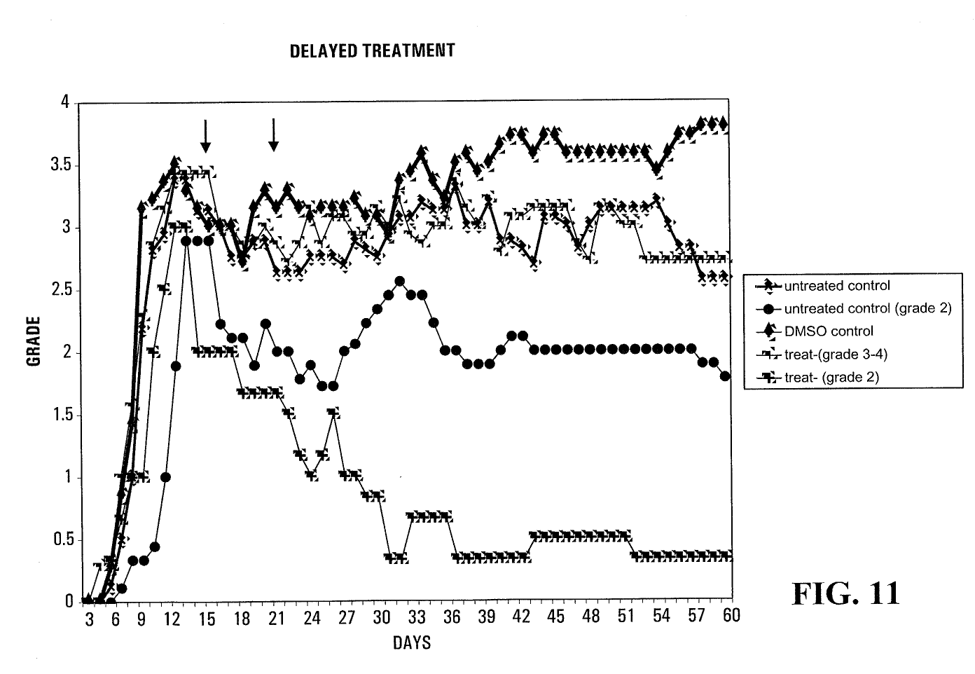

Blocking with cPLA₂ inhibitor prevents further relapse: To assess if cPLA₂ plays an important role in the progression of EAE, it was blocked using the chemical inhibitor AACOCF₃ described above. EAE induced C57BL/6 mice were given a delayed treatment with the cPLA₂ inhibitor on the day the animals began to remit (day 14). The animals were given a one-week treatment ( indicated by arrows in FIG. 11) and were monitored in a blind fashion using the clinical scoring scale described above. The treated animals could be divided into two groups: those that received treatment starting at day 14 that were at clinical grades of 3 and 4 , and those that were at a grade of 2. The former group showed a chronic/primary progressive form of the disease and were unaffected by the treatment regime and were not different from control groups. These groups peaked at a mean clinical score of 3.5. The animals in the treated and untreated control groups fell to a grade 2.6 and cycled back up to a score of 3.3. Vehicle treated animals progressed to a more severe form, reaching a mean clinical score of 3.8 (FIG. 11). In contrast, animals that received treatment on day 14 that had a clinical score of 2 had a remarkable reduction in the progression of the disease. Although these animals peaked to a mean clinical score of grade 3 prior to treatment, they progressively dropped down to a grade 0.3 (FIG. 11). Their advance into a second relapse was prevented. In contrast, untreated control animals that also showed a clinical score of 2 on day 14, peaked to a score of about 3.0, then remitted to a score of between 1-2 but progressed into a second paralytic episode (score of 2.5)and remained thereafter at a score of about 2.0. This clinical picture indicates a relapsing/remitting form of the disease. These results therefore provide very strong evidence that initiating treatments to block cPLA₂ after the peak of the first paralytic episode can prevent the occurrence of subsequent paralytic episodes in relapsing/remitting forms of the disease. It is possible that the chronic/primary progressive forms of EAE could be alleviated by higher doses or more prolonged treatment with the inhibitor.

All references cited herein or in the references section below are herein incorporated by reference.

References

Ackermann et al. (1995) Inhibition of macrophage Ca(2+)-independent phospholipase A2 by bromoenol lactone and trifluoromethyl ketones. J Biol Chem. 270:445-50.

Adelmann, M., Wood, J., Benzel, I., Fiori, P., Lassmann, H., Matthieu, J. M. et al., (1995). The N-terminal domain of the myelin oligodendrocyte glycoprotein (MOG) induces acute demyelinating experimental autoimmune encephalomyelitis in the Lewis rat. J. Neuroimmunol. 63: 17-27.

Allen, I., and Brankin, B. (1993). Pathogenesis of multiple sclerosis: the immune diathesis and the role of viruses. J. Neuropathol. Exp. Neurol. 52: 95-105.

Andersson P B, Goodkin D E. (1998) Glucocorticosteroid therapy for multiple sclerosis: a critical review. J Neurol Sci. 160(1):16-25.

Arbibe, L., Vial, D., Rosinski-Chupin, I., Havet, N., Huerre, M., Vargaftig, B. B. and Touqui, L. (1997). Endotoxin induces expression of type II phospholipase A2 in macrophages during acute lung injury in guinea pigs: involvement of TNF-alpha in lipopolysaccharide-induced type II phospholipase A2 synthesis. J. Immunol. 159: 391-400.

Balsinde, J., Balboa, M A., Insel, P A. And Dennis, E A (1999) Regulation and inhibition of phospholipase A₂. Annu. Rev. Pharmacol. Toxicol. 39: 175-189.

Bansil, S., Cook, S. D. and Rohowsky-Kochan, C. (1995). Multiple sclerosis: immune mechanism and update on current therapies. Ann. Neuol. 37 (S1):S87-S101.

Bauer J, Huitinga I, Zhao W, Lassmann H, Hickey W F, Dijkstra C D. (1995) The role of macrophages, perivascular cells, and microglial cells in the pathogenesis of experimental autoimmune encephalomyelitis. Glia. 4:437-46.

Bignami, A. and Ralston, H. J. III (1969). The cellular reaction to Wallerian degeneration in the central nervous system of the cat. Brain Res. 13: 444-461.

Bonventre, J. V., Huang, Z., Taheri, M. R., O'Leary, E., Li, E., Moskowitz, M. A. and Sapirstein, A. (1997). Reduced fertility and postischemic brain injury in mice deficient in cytosolic phospholipase A₂.

Brenner T, Boneh A, Shohami E, Abramsky O, Weidenfeld J. (1992) Glucocorticoid regulation of eicosanoid production by glial cells under basal and stimulated conditions. J Neuroimmunol. 40(2-3):273-9.

Brenner T, Brocke S, Szafer F, Sobel R A, Parkinson J F, Perez D H, Steinman L. (1997) Inhibition of nitric oxide synthase for treatment of experimental autoimmune encephalomyelitis. J Immunol. 158(6):2940-6.

Brenner T, Poradosu E, Soffer D, Sicsic C, Gazit A, Levitzki A. (1998) Suppression of experimental autoimmune encephalomyelitis by tyrphostin AG-556. Exp Neurol. 154(2):489-98.

Bruck., W., Schmied, M., Suchanek, G., Bruck, Y., Breitschopf, H., Poser, S., Piddlesden, S. and Lassmann, H. (1994). Oligodendrocytes in the early course of multiple sclerosis. Ann. Neuol. 35: 65-73.

Chang, H. W., Kudo, I., Tomita, M. and Inoue, K. (1987). Purification and characterization of extracellular phospholipase A2 from peritoneal cavity of caseinate-treated rat. J. Biochem. 102: 147-154.

Chen, S. and Bisby, M. A. (1993). Impaired motor axon regeneration in the C57/BL/Ola mouse. J. Comp. Neurol. 333:449-454.

Currie et al. (1994) Phosphorylation and activation of Ca(2+)-sensitive cytosolic phospholipase A2 in MCII mast cells mediated by high-affinity Fc receptor for IgE. Biochem J. 304:923-8.

Dennis, E. (1994) Diversity of group types, regulation, and function of phospholipase A2. J. Biol. Chem. 269: 13057-13060.

Dennis, E A (2000) Phopholipase A₂ in eicosanoid generation. Am. J. Respir. Crit. Care Med. 161: S32-S35.

Dinter H, Tse J, Halks-Miller M, Asarnow D, Onuffer J, Faulds D, Mitrovic B, Kirsch G, Laurent H, Esperling P, Seidelmann D, Ottow E, Schneider H, Tuohy V K, Wachtel H, Perez H D. (2000) The type IV phosphodiesterase specific inhibitor mesopram inhibits experimental autoimmune encephalomyelitis in rodents. J Neuroimmunol. 108(1-2):136-46.

Dore-Duffy, P., Donaldson, J. O., Koff, T., Longo, M. and Perry, W. (1986). Prostaglandin release in multiple sclerosis: correlation with disease activity. Neurology 36: 1587-1590.

Dore-Duffy, P., Ho, S. Y. and Donovan, C. (1991). Cerebrospinal fluid eicosanoid levels: endogenous PGD2 and LTC4 synthesis by antigen-presenting cells that migrate to the central nervous system. Neurology 41: 322-324.

Ebers, G. C. (1996). Genetic epidemiology of multiple sclerosis. Curr. Opinion Neurology 9: 155-158.

Emerson M R, Biswas S, LeVine S M. (2001) Cuprizone and piperonyl butoxide, proposed inhibitors of T-cell function, attenuate experimental allergic encephalomyelitis in SJL mice. J. Neuroimmunol. 119(2):205-13.

Ewing, C. and Bernard, C. C. (1998). Insights into the aetiology and pathogenesis of multiple sclerosis. Immunology & Cell Biology 76: 47-54.

Farooqui, A. A., Yang, H. C., Rosenberger, T. A. and Horrocks. L. A. (1997). Phospholipase A₂ and its role in brain tissue. J. Neurochem. 69: 889-901.

Fife B T, Kennedy K J, Paniagua M C, Lukacs N W, Kunkel S L, Luster A D, Karpus W J. (2001) CXCL10 (IFN-gamma-inducible protein-10) control of encephalitogenic CD4+ T cell accumulation in the central nervous system during experimental autoimmune encephalomyelitis. J Immunol. 166(12):7617-24.

Forst, S., Weiss, J., Elsbach, P., Maraganore, J. M., Reardon, I. and Heinrikson, R. L. (1986). Structural and functional properties of a phospholipase A2 purified from an inflammatory exudate. Biochemistry 25: 8381-8385.

Fretland D J. (1992) Potential role of prostaglandins and leukotrienes in multiple sclerosis and experimental allergic encephalomyelitis. Prostaglandins Leukot Essent Fatty Acids.45(4):249-57.

Fujimori, Y., Murakami, M., Kim, D. k., Hara, S. et al., (1992). Immunochemical detection of arachidonoyl-preferential phospholipase A2. J. Biochem. 111: 54-60

Gallai V, Sarchielli P, Trequattrini A, Franceschini M, Floridi A, Firenze C, Alberti A, Di Benedetto D, Stragliotto E. (1995) Cytokine secretion and eicosanoid production in the peripheral blood mononuclear cells of MS patients undergoing dietary supplementation with n-3 polyunsaturated fatty acids. J Neuroimmunol. 56(2):143-53.

Glaser, K. B., Mobilio, D., Chang, J. Y. and Senko, N. (1993). Phospholipase A₂ enzymes: regulation and inhibition. Trends in Pharmacol. 14: 92-98.

Goppelt-Struebe, M. and Rehfeldt, W. (1992). Glucocorticoids inhibit TNF alpha-induced cytosolic phospholipase A2 activity. Biochem Biophys Acta 1127: 163-167.

Goverman, J., Woods, A., Larson, L., et al., (1993). Transgenic mice that express a myelin basic protein-specific T-cell receptor develop spontaneous autoimmunity. Cell 72: 551-560.

Hall, S. M. (1989). Regeneration in the peripheral nervous system. Neuropathol. & Appl. Neurobiol. 15: 513-530.

Hall, S. M. (1993). Observations on the progress of Wallerian degeneration in transected peripheral nerves of C57BL/Wld mice in the presence of recruited macrophages. J. Neurocytol. 22:480-490.

Harlow, E. and Lane, D (1988) Antibodies: A Laboratory Manual, Cold Spring Harbor Laboratory Press, Cold Spring Harbor, N.Y.

Hauser, S. L., Doolittle, T. H., Lincoln, R., Brown, R. H. and Dinarello, C. A. (1990). Cytokine accumulations in CSF of multiple sclerosis patients: frequent detection of interleukin-1 and tumor necrosis factor but not interleukin-6. Neurology 40: 1735-1739.

Hirashima, Y., Farooqui, A. A., Mills, J. S. and Horrocks, L. A. (1992). Identification and purification of calcium-independent phopholipase A₂ from bovine brain cytosol. J. Neurochem. 59: 708-714.

Hofman, F. M., von Hanwehr, R. I., Dinarello, C. A., et al., (1986). Immunoregulatory molecules and IL-2 receptors identified in multiple sclerosis brain. J. Immunol. 136: 3239-3245.

Hofman, F. M., Hinton, D. R., Johnson, K., Merrill, J. E. (1989). Tumor necrosis factor identified in multiple sclerosis brain. J. Exp. Med. 170: 607-612.

Huitinga I, van Rooijen N, de Groot C J, Uitdehaag B M, Dijkstra C D. (1990) Suppression of experimental allergic encephalomyelitis in Lewis rats after elimination of macrophages. J Exp Med. 172(4):1025-33.

Hulkower, K. I., Hope, W. C., Chen, T., Anderson, C. M., Coffey, J. W. and Morgan, D. W. (1992). Interleukin-1 beta stimulates cytosolic phospholipase A2 in rheumatoid synovial fibroblasts. Biochem Biophys Res Commun. 184: 712-718.

Huterer, S. J., Tourtellotte, W. W. and Wherrett, J. R. (1995). Alterations in the activity of phospholipase A2 in postmortem white matter from patients with multiple sclerosis. Neurochem. Res. 20: 1335-1343.

Ishizaki, J., Ohara, O., Nakamura, E., Tamaki, M. et al., (1989). cDNA cloning and sequence determination of rat membrane-associated phospholipase A2. Biochem Biophys Res Commun 162: 1030-1036.

Issazadeh, S., Lorentzen, J. C., Mustafa, M. I., Hojeberg, B., Mussener, A. and Olsson, T. (1996). Cytokines in relapsing experimental autoimmune encephalomyelinitis in Da rats: persistent mRNA expression of proinflammatory cytokines and absent expression of interleukin-10 and transforming growth factor-beta. J. Neuroimmunol. 69: 103-115.

Ito A, Bebo B F Jr, Matejuk A, Zamora A, Silverman M, Fyfe-Johnson A, Offner H. (2001) Estrogen treatment down-regulates TNF-alpha production and reduces the severity of experimental autoimmune encephalomyelitis in cytokine knockout mice. J Immunol. 167(1):542-52.

Jeffery, N. D. and Blakemore, W. F. (1995). Remyelination of mouse spinal cord axons demyelinated by local injection of lysolecithin. J. Neurocytol. 24: 775-781.

Johns, T. G., Kerlero de Rosbo, N., Menon, K. K., Abo, S., Gonzales, M. F. and Bernard, C. C. (1995). Myelin oligodendrocytes glycoprotein induces a demyelinating encephalomyelitis resembling multiple sclerosis. J. Immnol. 154: 5536-3341.

Karpus W J, Lukacs N W, McRae B L, Strieter R M, Kunkel S L, Miller S D. (1995) An important role for the chemokine macrophage inflammatory protein-1 alpha in the pathogenesis of the T cell-mediated autoimmune disease, experimental autoimmune encephalomyelitis. J Immunol. 155(10):5003-10.

Kawai K, Kobayashi Y, Shiratori M, Sobue G, Tamatani T, Miyasaka M, Yoshikai Y (1996) Intrathecal administration of antibodies against LFA-1 and against ICAM-1 suppresses experimental allergic encephalomyelitis in rats. Cell Immunol. 171(2) :262-8.

Kennedy, B. P., Payette, P., Mudgett, J., Vadas, P., Pruzaniski, W., Kwan, M., Tang, C., Rancourt, D. E., and Cromlish, W. A. (1995). A natural disruption of group II PLA₂ gene in inbred mouse strains. J. Biol. Chem. 270: 22378-22385.

Kent S J, Karlik S J, Cannon C, Hines D K, Yednock T A, Fritz L C, Horner H C. (1995) A monoclonal antibody to alpha 4 integrin suppresses and reverses active experimental allergic encephalomyelitis. J Neuroimmunol. 58(1):1-10.

Klivenyl, P., Beal, M. F., Ferrante, R. J., Andreassen, O. A., Wermer, M., Chin, M -R. and Bonventre, J. V. 1998). Mice deficient in group IV cytosolic phospholipase A₂ are resistent to MPTP neurotoxicity. J. Neurochem. 71: 2634-2637.

Kohler G. and Milstein C. (1975) Continuous cultures of fused cells secreting antibody of predefined specificity. Nature 256:495-497.

Komada, M., Kudo, I., Mizushima, H., Kitamura, N. and Inoue, K. (1989). Structure of cDNA coding for rat platelet phospholipase A2. J. Biochem. 106: 545-547.

Kramer, R. M., Hession, C., Johansen, B., Hayes, G. et al., (1989). Structure and properties of a human non-pancreatic phospholipase A2. J. Biol. Chem. 264: 5768-5775.

Lautens, L. L., Chiou, X. G., Sharp, J. D., Young, W. S., Sprague, D. L., Ross, L. S. and Felder, C. C. (1998). Cytolsolic phospholipase A₂ (cPLA₂) distribution in murine brain and functional studies indicate that cPLA₂ does not participate in muscarinic receptor-mediate signalling in neurons. Brain Res. 809: 18-30.

Lee, S. J. and Benveniste, E. N. (1999). Adhesion molecule expression and regulation on cells of the central nervous system. J. Neuroimmunol. 98: 77-88.

Leonard J P, Waldburger K E, Goldman S J. (1996) Regulation of experimental autoimmune encephalomyelitis by interleukin-12. Ann N Y Acad Sci. 795:216-26.

Lin, L. L., Lin, A. Y. and DeWitt, D. L. (1992). Interleukin-1 alpha induces the accumulation of cytosolic phospholipase A2 and the release of prostaglandin E2 in human fibroblasts. J. Biol. Chem. 267: 23451-23454.

Lin, L. L., Wartmann, M., Lin, A. Y., Knopf, J. L., Seth, A. and Davis, R. J. (1993). cPLA2 is phosphorylated and activated by MAP kinase. Cell 72: 269-278.

Liu, J., Marino, M. W., Wong, G., Grail, D., Dunn, A., Bettadapura, J., Slavin, A. J., Old, L. and Bernard, C. C. (1998). TNF is a potent anti-inflammatory cytokine in autoimmune-mediated demyelination. Nature Medicine 4: 78-8326.

Lyons-Giordano, B., Davis, G. L., Galbraith, W., Pratta, M. A. and Arner, E. C. (1989). Interleukin-1 beta stimulates phospholipase A2 mRNA synthesis in rabbit articular chondrocytes. Biochem Biophys Res Commun. 164: 488-495.

Martin, R., Jaraquemada, D., Flerlage, M., et al., (1990). Fine specificity and HLA restriction of myelin basic protein-specific cytotoxic T-cell lines from multiple sclerosis patients and heathy individuals. J. Immunol. 145: 540-548.

Meyer A L, Benson J M, Gienapp I E, Cox K L, Whitacre C C. (1996) Suppression of murine chronic relapsing experimental autoimmune encephalomyelitis by the oral administration of myelin basic protein. J Immunol. 157(9):4230-8.

Mokhtarian, F., McFarlin, D. E. and Raine, C. S. (1984). Adoptive transfer of myelin basic protein-sensitive T cells produces chronic relapsing demyelinating disease in mice. Nature 309: 356-358.

Molloy, G. Y., Rattray, M. and Williams, R. J. (1998). Genes encoding multiple forms of phospholipase A₂ are expressed in rat brain. Neurosci. Lett. 258: 139-142.

Murakami, M., Kudo, I., Natori, Y. and Inoue, K. (1990). Immunochemical detection of □platelet type□ phospholipase A2 in the rat. Biochem Biophys Acta 1043: 34-42.

Murakami, M., Kudo, I. and Inoue, K. (1993). Molecular nature of phospholipase A2 involved in prostaglandin 12 synthesis in human umbilical vein endothelial cells. Possible participation of cytosolic and extracellular type II phospholipase A2. J. Biol. Chem. 268: 839-844

Murakami, M., Nakatani, Y., Atsumi, G., Inoue, K. and Kudo, I. (1997). Regulatory functions of phospholipase A₂. Critical Reviews in Immunol. 17: 225-283.

Nalefski, E. A., Sultzman, L. A., Martin, D. M., et al. (1994) Delineation of two functionally distinct domains of cytosolic phopholipase A2, a regulatory Ca(2+)-dependent lipid binding domain and a Ca(2+)-independent catalytic domain. J. Biol. Chem. 269:1823-1849.

Nakano, T. and Arita, H. (1990). Enhanced expression of group II phospholipase A2 gene in the tissues of endotoxin shock rat and its suppression by glucocorticoid. FEBS Let. 273: 23-26.

Nakano, T., Ohara, O., Teraoka, H. and Arita, H. (1990). GroupII phospholipase A2 mRNA synthesis is stimulated by two distinct mechanisms in rat vascular smooth muscle cells. FEBS Lett. 261: 171-174.

Norton, W. T., Brosnan, C. F., Cammer, W. and Goldmuntz, E. A. (1990). Mechanisms and suppression of inflammatory demyelination. Acta Neuropathologiae Experimentalis 50: 225-235.

Noseworthy J H, Lucchinetti C, Rodriguez M, Weinshenker B G. (2000) Multiple sclerosis. N Engl J Med. 28;343(13):938-52.

Ogata, K., Jin, B., Taniguchi, M., et al., (2001) Attenuation of ishemicia and reperfusion injury of canine livers by inhibition of type II phospholipase A₂ with LY329722. Transplantation 71: 1040-1046.

Oger, J. and Lai, H. (1994). Demyelination and ethnicity: experience at the University of British Columbia Multiple Sclerosis Clinic with special reference to HTLV-1-associated myelopathy in British Columbian natives. Ann. Neurol. 36: S22-24.

Oka, S. and Arita, H. (1991). Inflammatory factors stimulate expression of group II phospholipase A2 in rat cultured astrocytes. Two distinct pathways of the gene expression. J. Biol. Chem. 266: 9956-9960.

Olsson, T., Wei, Z. H., Hojeberg, B. et al., (1990). Autoreactive T lymphocytes in multiple sclerosis determined by antigen-induced secretion of interferon-□. J. Clin. Invest. 86: 981-985.

Oinuma et al. (1991) Synthesis and biological evaluation of substituted benzenesulfonamides as novel potent membrane-bound phospholipase A2 inhibitors. J Med Chem. 34:2260-7.

Ousman, S. and David, S. (2000). Lysophosphatidylcholine induces rapid recruitment and activation of macrophages in the adult mouse spinal cord. Glia 30: 92-104.

Ousman, S. and David, S. (2001). MIP-1α, GM-CSF, and TNF-α control the immune cell response that mediates rapid phagocytosis of myelin from the adult mouse spinal cord. J. Neurosci. 21: 4649-4656.

Owada, Y., Tominaga, T., Yoshimoto, T. and Kondo, H. (1994). Molecular cloning of rat cDNA for cytosolic phospholipase A₂ and the increased gene expression in the dentate gyrus following transient forebrain ischemia. Mol. Brain Res. 25: 364-368.

Ozawa, K., Suchanek, G., Breitschopf, H., Bruck, W., Budka, H., Jellinger, K. and Lassmann, H. (1994). Patterns of oligodendroglia pathology in multiple sclerosis. Brain 117: 1311-1322.

Panitch, H. S., Hirsch, R. L., Schindler, J., et al., (1987). Treatment of multiple sclerosis with gamma interferon: exacerbations associated with activation of the immune system. Neurology 37: 1097-1102.

Penkowa M, Hidalgo J. (2000) Metallothionein I+II expression and their role in experimental autoimmune encephalomyelitis. Glia.32(3):247-63.

Pitt D, Werner P, Raine C S. (2000) Glutamate excitotoxicity in a model of multiple sclerosis. Nat Med. Jan;6(1):67-70.

Polman C H, Uitdehaag B M. (2000) Drug treatment of multiple sclerosis. West J Med. 173(6):398-402.

Popovich P G, Yu J Y, Whitacre C C. (1997) Spinal cord neuropathology in rat experimental autoimmune encephalomyelitis: modulation by oral administration of myelin basic protein. J Neuropathol Exp Neurol. 56(12):1323-38.

Prineas, J. W., Barnard, R. O., Kwon, E. E., et al., (1993). Multiple sclerosis: remyelination of nascent lesions. Ann. Neurol. 33: 137-151.

Probert, L., Akassoglou, K., Pasparakis, M., Kontogeorgos, G. and Kollias, G. (1995). Spontaneous inflammatory demyelinating disease in transgenic mice showing central nervous system-specific expression of tumor necrosis factor alpha. Proc. Natl. Acad. Sci. USA 92: 11294-11298.

Prokazova, N. V., Zvezdina, N. D. and Korotaeva, A. A. (1998). Effect of lysophosphatidylcholine on transmembrane signal transduction. Biochemistry 63: 31-37.

Prosiegel M, Neu I, Mallinger J, Wildfeuer A, Mehlber L, Vogl S, Hoffmann G, Ruhenstroth-Bauer G. (1989) Suppression of experimental autoimmune encephalomyelitis by dual cyclo-oxygenase and 5-lipoxygenase inhibition. Acta Neurol Scand. 79(3):223-6.

Prosiegel M, Neu I, Vogl S, Hoffmann G, Wildfeuer A, Ruhenstroth-Bauer G. (1990) Suppression of experimental autoimmune encephalomyelitis by sulfasalazine. Acta Neurol Scand. 81(3):237-8.

Racke M K, Burnett D, Pak S H, Albert P S, Cannella B, Raine C S, McFarlin D E, Scott D E. (1995) Retinoid treatment of experimental allergic encephalomyelitis. IL-4 production correlates with improved disease course. J Immunol. 154(1):450-8.

Raine, C. S. and Cannella, B. (1992). Adhesion molecules and central nervous system inflammation. Semin. Neurosci. 4: 201-211.

Reder, A T., Thapur, M., Sapugay, A M and Jensen, A M. (1994) Prostaglandins and inhibitors of acrachidonate metabolism suppress experimental allergic encephalomyelitis. J.Neuroimmunol. 54: 117-127.

Reichert, J. R., Robinson, E. D., Deibler, G. E., et al., (1989). Human cytotoxic T-cell recognition of a synthetic peptide of myelin basic protein. Ann. Neuol. 26: 342-346.

Renno, T., Lin, J. Y., Piccirillo, C., Antel, J. and Owens, T. (1994). Cytokine production by cells in cerebrospinal fluid during experimental allergic encephalomyelitis in SJL/J mice. J. Neuroimmunol. 49: 1-7.

Reynolds et al. (1994) 1-Hexadecyl-2-arachidonoylthio-2-deoxy-sn-glycero-3-phosphorylcholine as a substrate for the microtiterplate assay of human cytosolic phospholipase A2. Anal Biochem 217:25-32.

Rolak L A. (2001) Multiple sclerosis treatment 2001. Neurol Clin. 19(1):107-18.

Ross, B. M., Kim, D. K., Bonventre, J. V. and Kish, S. J. (1995). Characterization of a novel phospholipase A₂ activity in human brain. J. Neurochem. 64: 2213-2221.

Ruddle N H, Bergman C M, McGrath K M, Lingenheld E G, Grunnet M L, Padula S J, Clark R B (1990) An antibody to lymphotoxin and tumor necrosis factor prevents transfer of experimental allergic encephalomyelitis. J Exp Med. 1990 172(4):1193-200.

Ruuls et al. (1996) J. Immunol. 157:5721-5731.

Ryborg, A. K., Deleuran, B., Thestrup-Pedersen, K. and Kragballe, K. (1994). Lysophosphatidylcholine: a chemoattractant to human T lymphocytes. Arch. Dermatol. Res. 286: 462-465.

Sadovnick, A. D., Ebers, G. C., Dyment, D. A. and Risch, N. J. (1996). Evidence for genetic basis of multiple sclerosis. The Canadian Collaborative Study Group. Lancet 347: 1728-1730.

Seilhamer, J. J., Pruzanski, W., Vadas, P., Plant, S. et al., (1989). Cloning and recombinant expression of phospholipase A2 present in rheumatoid arthritis synovial fluid. J. Biol. Ed Chem. 264: 5335-5338.

Selmaj K, Walczak A, Mycko M, Berkowicz T, Kohno T, Raine C S. (1998) Suppression of experimental autoimmune encephalomyelitis with a TNF binding protein (TNFbp) correlates with down-regulation of VCAM-1/VLA-4. Eur J Immunol. 28(6):2035-44.

Sharp, J. D., White, D. L., Chiou, X. G. et al. (1991) Molecular Cloning and Expression of human Ca²+-sensitive cytosolic phospholipase A₂. J. Biol. Chem. 266: 14850-14853.

Smith T, Groom A, Zhu B, Turski L. (2000) Autoimmune encephalomyelitis ameliorated by AMPA antagonists. Nat Med. 6(1):62-6.

Sobel, R. A., Mitchell, M. E. and Fondren, G. (1990). Intracellular adhesion molecule-1 (ICAM-1) in cellular immune reactions in the human central nervous system. Am. J. Pathol. 136: 1309-1316.

Steinman L, Miller A, Bernard C C, Oksenberg J R. (1994). The epigenetics of multiple sclerosis: clues to etiology and a rationale for immune therapy. Annu Rev Neurosci. 17:247-65.

Stinissen P, Raus J, Zhang J. (1997) Autoimmune pathogenesis of multiple sclerosis: role of autoreactive T lymphocytes and new immunotherapeutic strategies. Crit Rev Immunol. 17(1):33-75.

Street et al. (1993) Slow- and tight-binding inhibitors of the 85-kDa human phospholipase A2. Biochemistry 32:5935-40.

Sun, J. B., Olsson, T., Wang, W. Z., et al., (1991). Autoreactive T and B cells responding to myelin proteolipid protein in multiple sclerosis and controls. Eur. J. Immunol. 21: 1461-1468.

Taupin, V., Renno, T., Bourbonniere, L., Peterson, A. C., Rodriguez, M. and Owens, T. (1997). Increased severity of experimental autoimmune encephalomyelitis, chronic macrophage/microglial reactivity, and demyelination in transgenic mice producing tumor necrosis factor-alpha in the central nervous system. Eurp. J. Immunol. 27: 905-913.

Tienari, P. (1994). Multiple sclerosis: multiple etiologies, multiple genes? Annals of Med. 26: 259-269.

Tran E H, Hoekstra K, van Rooijen N, Dijkstra C D, Owens T. (1998) Immune invasion of the central nervous system parenchyma and experimental allergic encephalomyelitis, but not leukocyte extravasation from blood, are prevented in macrophage-depleted mice. J Immunol. 161(7):3767-75.

Traugott, U., Reinherz, E. L., Raine, C. S. (1983). Multiple sclerosis: distribution of T cell subsets within active chronic lesion. Science 219: 308-310.

Trotter, J. and Smith, M. E. (1986). The role of phospholipases from inflammatory macrophages in demyelination. Neurochem. Res. 11: 349-361.

Tuohy, V. K. (1994). Peptide determinants of myelin proteolipid protein (PLP) in autoimmune demyelinating disease: a review. Neurochemical Res. 19: 935-944.

van der Meide et al. (1998) J. Neuroimmunol. 84:14-23.

Warren, K. G., Catz, I., Johnson, E. and Mielke, B. (1994). Anti-myelin basic protein and anti-proteolipid protein specific forms of multiple sclerosis. Ann. Neurol. 35: 280-289.

Weber F, Hempel K. (1989) Protection against experimental allergic encephalomyelitis with complete Freund's adjuvant is unaffected by prostaglandin synthesis inhibition. Int Arch Allergy Appl Immunol. 89(2-3):242-5.

Woelk, H. and Peiler-Ichikawa, K. (1974) On the activity of phospholipase A₂ compared with 1-alk-1′-enyl-2-acyl and 1-alkyl-2-glcerophosphatides in multiple sclerosis. J. Neurol. 207: 319-326.

Wright, G. W., Ooi, C. E., Weiss, J. and Elsbach, P. (1989). Purification of a cellular (granulocyte) and an extracellular (serum) phospholipase A2 that participate in the destruction of Escherichia coli in a rabbit inflammatory exudate. J. Biol. Chem. 265: 6675-6681.

Wu, T., Levine, S. J., Lawrence, M. G., Logun, C., Angus, C. W. and Shehamer, J. H. (1994). Interferon-gamma induces the synthesis and activation of cytosolic phospholipase A₂. J. Clin. Invest. 93: 571-577.

Xu, X. X., Rock, C. O., Qui, Z. H., Leslie, C. C. and Jackowski, S. (1994). Regulation of cytosolic phospholipase A2 phosphorylation and eicosanoid production by colony-stimulating factor-1. J. Biological Chem. 269: 31693-31700.

Yang, H -C., Farooqui, A. A., and Horrocks, L. A. (1994). Effects of glycosaminoglycans and glycosphigolipids on cytolsolic phospholipase A₂ from bovine brain. Biochem. J. 299: 91.

Yelton D. E. and Scharff M. D. (1981) Monoclonal Antibodies: a powerful new tool in biology and medicine. Ann. Rev. Biochem. 50:657-680.

Yednock T A, Cannon C, Fritz L C, Sanchez-Madrid F, Steinman L, Karin N (1992) Prevention of experimental autoimmune encephalomyelitis by antibodies against alpha 4 beta 1 integrin. Nature 356(6364):63-6.

Yu M, Nishiyama A, Trapp B D, Tuohy V K. (1996) Interferon-beta inhibits progression of relapsing-remitting experimental autoimmune encephalomyelitis. J Neuroimmunol. 64(1):91-100.

Zamvil, S., Nelson, P., Trotter, J., Mitchell, D., Knobler, R., Fritz, R. and Steinman, L. (1985). T-cell clones specific for myelin-basic protein induce chronic relapsing paralysis and demyelination. Nature 317: 355-358.

Zhang, Y., Burger, D., Saruhan, G., et al., (1993). The lymphocyte response against myelin-associated glycoprotein and myelin basic protein in patients with multiple sclerosis. Neurology 43: 403-407.

1. A method of preventing or treating a neural inflammatory or demyelinating disease in an animal, said method comprising inhibiting the activity of a phospholipase A₂ in the animal.

2. The method of claim 1, wherein the animal is a mammal.

3. The method of claim 1, wherein the animal is a human.

4. The method of claim 1, wherein the neural inflammatory or demyelinating disease is Multiple Sclerosis.

5. The method of claim 1, wherein the phospholipase A₂ is a cytosolic phospholipase A₂.

6. The method of claim 1, wherein the method comprises administering to the animal an effective amount of a phospholipase A₂ inhibitor.

7. The method of claim 1, wherein the inhibitor is selected from the group consisting of arachidonic acid analogues, benzenesulfonamide derivatives, bromoenol lactone, p-bromophenyl bromide, bromophenacyl bromide, trifluoromethylketones, sialoglycolipids and proteoglycans.

8. The method of claim 7, wherein the inhibitor is selected from the group consisting of arachidonyl trifluoromethyl ketone, methyl arachidonyl fluorophosphonate, palmitoyl trifluoromethyl ketone.

9. The method of claim 1, wherein the method comprises inhibiting the expression of a phospholipase A₂.

10. The method of claim 9, wherein the method comprises administering to the animal an effective amount of a phospholipase A2 inhibitor.

11. The method of claim 10, wherein the phospholipase A₂ inhibitor is an antisense molecule.

12. The method of claim 11 wherein the antisense molecule is a nucleic acid that is substantially complementary to a portion of an mRNA encoding a phospholipase A₂.

13. The method of claim 12 wherein the antisense molecule is complementary to a portion of a nucleic acid sequence substantially identical to a sequence selected from the group consisting of SEQ ID NO. 1 and SEQ ID NO. 3.

14. The method of claim 12 wherein the portion of an mRNA comprises at least 5 contiguous bases.

15. A method for identifying and/or characterizing a compound for the prevention or treatment of a neural inflammatory or demyelinating disease, said method comprising assaying the activity or expression of a phospholipase A₂ in the presence of a test compound, to identify a compound that inhibits phospholipase A₂ activity or expression, wherein inhibition is indicative that the test compound may be useful for the prevention or treatment of a neural inflammatory or demyelinating disease.

16. The method of claim 15, further comprising the step of assaying the compounds for activity in the prevention or treatment of a neural inflammatory or demyelinating disease.

17. The method of claim 15, wherein the phospholipase A₂ is a mammalian phospholipase A₂.

18. The method of claim 17, wherein the phospholipase A₂ is a human phospholipase A₂.

19. The method of claim 15, wherein the phospholipase A₂ is a cytosolic phospholipase A₂.

20. The method of claim 15, wherein the neural inflammatory or demyelinating disease is Multiple Sclerosis.

21. A method of assessing a neural inflammatory or demyelinating disease in an animal, said method comprising:
(a) determining a test level of phospholipase A₂ protein or phospholipase A₂ encoding mRNA or phospholipase A₂ enzyme activity in tissue or body fluid of the animal; and
(b) comparing said test level of phospholipase A₂ protein or phospholipase A₂ encoding mRNA or phospholipase A₂ activity to an established standard; or to a corresponding level of phospholipase A₂ protein or phospholipase A₂ encoding mRNA or phospholipase A₂ enzyme activity in tissue or body fluid of a control animal; or to a corresponding level of phospholipase A₂ protein or phospholipase A₂ encoding mRNA or phospholipase A₂ enzymatic activity in tissue or body fluid obtained from said animal at an earlier time;
wherein an increase in said test level is indicative of the neural inflammatory or demyelinating disease.

22. The method of claim 21, wherein the animal is a mammal.

23. The method of claim 22, wherein the animal is a human.

24. The method of claim 21, wherein the phospholipase A₂ is a cytosolic phospholipase A₂.

25. The method of claim 21, wherein the neural inflammatory or demyelinating disease is Multiple Sclerosis.

26. The method of claim 21, wherein the tissue or body fluid is selected from the group consisting of blood, plasma, cerebrospinal fluid, endothelia, macrophages and lymphocytes.


SEQUENCE LISTING (5 sequences)

SEQ ID NO: 1 - DNA, 2846 bp, Homo sapiens
  CDS (126)..(2375)
ctgaaaaaggatcctgactgaaagctagaggcattgaggagcctgaagattctcaggttttaaagacgctagagtgccaaagaagactttgaagtgtgaaaacatttcctgtaattgaaaccaaaatgtcatttatagatccttaccagcacattatagtggagcaccagtattcccacaagtttacggtagtggtgttacgtgccaccaaagtgacaaagggggcctttggtgacatgcttgatactccagatccctatgtggaactttttatctctacaacccctgacagcaggaagagaacaagacatttcaataatgacataaaccctgtgtggaatgagacctttgaatttattttggatcctaatcaggaaaatgttttggagattacgttaatggatgccaattatgtcatggatgaaactctagggacagcaacatttactgtatcttctatgaaggtgggagaaaagaaagaagttccttttattttcaaccaagtcactgaaatggttctagaaatgtctcttgaagtttgctcatgcccagacctacgatttagtatggctctgtgtgatcaggagaagactttcagacaacagagaaaagaacacataagggagagcatgaagaaactcttgggtccaaagaatagtgaaggattgcattctgcacgtgatgtgcctgtggtagccatattgggttcaggtgggggtttccgagccatggtgggattctctggtgtgatgaaggcattatacgaatcaggaattctggattgtgctacctacgttgctggtctttctggctccacctggtatatgtcaaccttgtattctcaccctgattttccagagaaagggccagaggagattaatgaagaactaatgaaaaatgttagccacaatccccttttacttctcacaccacagaaagttaaaagatatgttgagtctttatggaagaagaaaagctctggacaacctgtcacctttactgatatctttgggatgttaataggagaaacactaattcataatagaatgaatactactctgagcagtttgaaggaaaaagttaatactgcacaatgccctttacctcttttcacctgtcttcatgtcaaacctgacgtttcagagctgatgtttgcagattgggttgaatttagtccatacgaaattggcatggctaaatatggtacttttatggctcccgacttatttggaagcaaattttttatgggaacagtcgttaagaagtatgaagaaaaccccttgcatttcttaatgggtgtctggggcagtgccttttccatattgttcaacagagttttgggcgtttctggttcacaaagcagaggctccacaatggaggaagaattagaaaatattaccacaaagcatattgtgagtaatgatagctcggacagtgatgatgaatcacacgaacccaaaggcactgaaaatgaagatgctggaagtgactatcaaagtgataatcaagcaagttggattcatcgtatgataatggccttggtgagtgattcagctttattcaataccagagaaggacgtgctgggaaggtacacaacttcatgctgggcttgaatctcaatacatcttatccactgtctcctttgagtgactttgccacacaggactcctttgatgatgatgaactggatgcagctgtagcagatcctgatgaatttgagcgaatatatgagcctctggatgtcaaaagtaaaaagattcatgtagtggacagtgggctcacatttaacctgccgtatcccttgatactgagacctcagagaggggttgatctcataatctcctttgacttttctgcaaggccaagtgactctagtcctccgttcaaggaacttctacttgcagaaaagtgggctaaaatgaacaagctcccctttccaaagattgatccttatgtgtttgatcgggaagggctgaaggagtgctatgtctttaaacccaagaatcctgatatggagaaagattgcccaaccatcatccactttgttctggccaacatcaacttcagaaagtacaaggctccaggtgttccaagggaaactgaggaagagaaagaaatcgctgactttgatatttttgatgacccagaatcaccattttcaaccttcaattttcaatatccaaatcaagcattcaaaagactacatgatcttatgcacttcaatactctgaacaacattgatgtgataaaagaagccatggttgaaagcattgaatatagaagacagaatccatctcgttgctctgtttcccttagtaatgttgaggcaagaagatttttcaacaaggagtttctaagtaaacccaaagcatagttcatgtactggaaacggcagcagtttctgatgctgaggcagtttgcaatcccatgacaactggatttaaaagtacagtacagatagtcgtactgatcatgagagactggctgatactcaaagttgcagttacttagctgcatgagaataatactattataagttaggttgacaaatgatgttgattatgtaaggatatacttagctacattttcagtcagtatgaacttcctgatacaaatgtagggatatatactgtatttttaaacatttctcaccaactttcttatgtgtgttctttttaaaaattttttttcttttaaaatatttaacagttcaatctcaataagacctcgcattatgtatgaatgttattcactgactagatttattcataccatgagacaacactatttttatttatatatgcatatatatacatacatgaaataaatacatcaatataaaaat

SEQ ID NO: 2 - protein, 749 aa, Homo sapiens
MSFIDPYQHIIVEHQYSHKFTVVVLRATKVTKGAFGDMLDTPDPYVELFISTTPDSRKRTRHFNNDINPVWNETFEFILDPNQENVLEITLMDANYVMDETLGTATFTVSSMKVGEKKEVPFIFNQVTEMVLEMSLEVCSCPDLRFSMALCDQEKTFRQQRKEHIRESMKKLLGPKNSEGLHSARDVPVVAILGSGGGFRAMVGFSGVMKALYESGILDCATYVAGLSGSTWYMSTLYSHPDFPEKGPEEINEELMKNVSHNPLLLLTPQKVKRYVESLWKKKSSGQPVTFTDIFGMLIGETLIHNRMNTTLSSLKEKVNTAQCPLPLFTCLHVKPDVSELMFADWVEFSPYEIGMAKYGTFMAPDLFGSKFFMGTVVKKYEENPLHFLMGVWGSAFSILFNRVLGVSGSQSRGSTMEEELENITTKHIVSNDSSDSDDESHEPKGTENEDAGSDYQSDNQASWIHRMIMALVSDSALFNTREGRAGKVHNFMLGLNLNTSYPLSPLSDFATQDSFDDDELDAAVADPDEFERIYEPLDVKSKKIHVVDSGLTFNLPYPLILRPQRGVDLIISFDFSARPSDSSPPFKELLLAEKWAKMNKLPFPKIDPYVFDREGLKECYVFKPKNPDMEKDCPTIIHFVLANINFRKYKAPGVPRETEEEKEIADFDIFDDPESPFSTFNFQYPNQAFKRLHDLMHFNTLNNIDVIKEAMVESIEYRRQNPSRCSVSLSNVEARRFFNKEFLSKPKA

SEQ ID NO: 3 - DNA, 2787 bp, Mus musculus
  CDS (109)..(2355)
ggcacagagaagcctgaggattctcatttaactctgggaactgcttcaagaagctacagtaccatagaagacctgggaagtgtgagaatttctgcaactgggaccaaaatgtctttcatagatccttatcagcacattatagtggaacaccagtactcccataagtttactgttgtggttctacgtgccaccaaagtaaccaaggggacctttggcgatatgctggacactccagatccttatgtggaacttttcatctctacaacccctgacagcaggaagcgaacgagacacttcaataatgatataaaccccgtgtggaatgagacctttgagttcattttggatcctaatcaggaaaatgttttggagatcacactgatggatgccaactacgtcatggatgaaaccctaggcacagctacattccctgtatcttcaatgaaagtgggagagaagaaagaagtcccttttattttcaaccaagtcactgaaatgattctggaaatgtctctggaagtttgttcatgcccagacctacggttcagcatggcactgtgtgatcaggagaaaactttcagacagcagaggaaagagaacataaaagagaacatgaagaaacttttgggtccaaaaaagagtgaggggctttattccacacgtgatgtgccggtggtggccattttgggttcaggtgggggtttccgggccatggtgggattctctggtgtgatgaaggcactgtatgagtcggggattttggactgtgctacatacattgctggtctttctggatccacatggtacatgtcaaccttgtactctcaccccgattttccagagaaaggtcccgaggagattaatgaagagctaatgaaaaatgtcagccacaaccctctcttacttcttacaccacagaaagttaaaagatacgttgagtctttatggaagaagaaaagttctggccagcctgtcacctttactgacatctttgggatgctaataggagaaacactaattcaaaataggatgagcatgaccctgagtagtttgaaggaaaaggtcaatgccgcccggtgtcctttgcctctcttcacgtgtctccacgtcaaacctgatgtgtcagagctgatgtttgccgattgggtggaatttagtccatatgagattggcatggcaaaatatggtacctttatggctcctgacctatttggaagcaagttttttatgggaacagttgtaaaaaaatatgaagaaaaccccttgcatttcttgatgggtgtctggggcagtgccttttctatactgttcaacagagttttgggagtttctggctcacagaataaaggctctacaatggaagaggaattagaaaatattacagcaaagcacatcgtgagtaatgacagctccgacagtgatgatgaggctcaaggacccaaaggcaccgagaatgaagaagctgaaaaagagtaccaaagcgacaaccaagcaagttgggtccatcggatgctaatggccttggtgagcgactcggctttattcaatacccgagaaggacgtgccggaaaggtgcataacttcatgctgggcttgaatctcaacacatcatatccactgtctcccctgagagacttcagctctcaggattccttcgatgacgagctcgacgcagcggtagcagatccagatgaatttgaacgaatatatgaaccactggatgtcaaaagtaagaagattcatgtggtagatagtgggctcacatttaacctgccatatcccttgattcttcgacctcagagaggtgtggatcttatcatctcctttgacttttctgcaaggccgagtgacaccagtccccctttcaaggaacttctgcttgcagagaagtgggcgaaaatgaacaagcttccctttccaaagatcgatccttatgtgtttgatcgggaaggattaaaggaatgctatgtttttaaacctaagaatcctgatgtggagaaggattgcccaaccattatccactttgttctggccaacatcaacttcagaaagtacaaggccccaggtgttctaagggaaaccaaagaagagaaagaaattgctgactttgacatttttgatgaccccgaatcgccattttcaaccttcaactttcagtatcccaatcaagcattcaaaaggcttcacgatttgatgtacttcaacacactgaacaacattgatgtgataaaggatgccattgttgagagcattgaatacagaagacagaacccatctcgttgctctgtttccctcagtaatgttgaagcaagaaaattcttcaataaggagtttctaagtaaacccactgtgtaatttctgtgctgggatgatcaagccatttgaattccatgacaatttgagttcagaagacattagaggtcatcttactatgcagaagagactggctgctactcaaagttgtggagatttagccatgtgttaggtgaaaatgatgttgattatgtaatacttagcaacagtttctgacagtatgaattttttgacattagcatagagctatatactgtattttaaacattcctcacattttttacctgtactttttatataaatatgacatgtcttttcttttgaaaatatttaatagtttaactcagtaaaggagacttcccattgtgtgtgaatgttattctgaactagatttgttcatgccatgttacaacactatttttatttaaatgtttatatttacacatacgaaataaatactttgctgtacaaatt

SEQ ID NO: 4 - protein, 748 aa, Mus musculus
MSFIDPYQHIIVEHQYSHKFTVVVLRATKVTKGTFGDMLDTPDPYVELFISTTPDSRKRTRHFNNDINPVWNETFEFILDPNQENVLEITLMDANYVMDETLGTATFPVSSMKVGEKKEVPFIFNQVTEMILEMSLEVCSCPDLRFSMALCDQEKTFRQQRKENIKENMKKLLGPKKSEGLYSTRDVPVVAILGSGGGFRAMVGFSGVMKALYESGILDCATYIAGLSGSTWYMSTLYSHPDFPEKGPEEINEELMKNVSHNPLLLLTPQKVKRYVESLWKKKSSGQPVTFTDIFGMLIGETLIQNRMSMTLSSLKEKVNAARCPLPLFTCLHVKPDVSELMFADWVEFSPYEIGMAKYGTFMAPDLFGSKFFMGTVVKKYEENPLHFLMGVWGSAFSILFNRVLGVSGSQNKGSTMEEELENITAKHIVSNDSSDSDDEAQGPKGTENEEAEKEYQSDNQASWVHRMLMALVSDSALFNTREGRAGKVHNFMLGLNLNTSYPLSPLRDFSSQDSFDDELDAAVADPDEFERIYEPLDVKSKKIHVVDSGLTFNLPYPLILRPQRGVDLIISFDFSARPSDTSPPFKELLLAEKWAKMNKLPFPKIDPYVFDREGLKECYVFKPKNPDVEKDCPTIIHFVLANINFRKYKAPGVLRETKEEKEIADFDIFDDPESPFSTFNFQYPNQAFKRLHDLMYFNTLNNIDVIKDAIVESIEYRRQNPSRCSVSLSNVEARKFFNKEFLSKPTV

SEQ ID NO: 5 - protein, 21 aa, Mus musculus
MEVGWYRSPFSRVVHLYRNGK